(19) United States
(12) Patent Application Publication
(10) Pub. No.: US 2003/0170173 A1
(43) Pub. Date: Sep. 11, 2003

(54) Contrast agents

(76) Inventors: Jo Klaveness, Oslo (NO); Helge Tolleshaug, Oslo (NO); Alan Cuthbertson, Oslo (NO); Mari Ann Kulseth, Oslo (NO)

Correspondence Address: Amersham Biosciences Corp, 800 Centennial Avenue, Piscataway, NJ, 08855, US

(21) Appl. No.: 10/302,419

(22) Filed: Nov. 22, 2002

Related U.S. Application Data
(63) Continuation of application No. PCT/NO01/00215, filed on May 23, 2001.
(60) Provisional application No. 60/210,061, filed on Jun. 7, 2000.

(30) Foreign Application Priority Data
May 23, 2000 (NO) 20002644

(51) Int. Cl. A61K 51/00; A61K 49/04

(52) U.S. Cl. 424/1.11; 424/9.3; 424/9.4; 424/9.6

(57) ABSTRACT

This invention relates to contrast agents and the use of these contrast agents for diagnosis of diseases in humans and animals based on mapping of metabolic activity. The contrast agents can be used to identify tissue or cells with metabolic activity or enzymatic activity deviating from the normal. A contrast agent substrate changes pharmacodynamic and/or pharmacokinetic properties upon a chemical modification from a contrast agent substrate to a contrast agent product in a specific enzymatic transformation, thereby detecting areas of disease upon a deviation in the enzyme activity from the normal.

This invention relates to contrast agents and the use of these contrast agents in diagnosis of diseases in humans and animals based on mapping of metabolic activity. The contrast agents can be used to identify tissue or cell(s) with reduced metabolic or enzymatic activity or more preferably to identify tissue or cell(s) with increased metabolic or enzymatic activity.

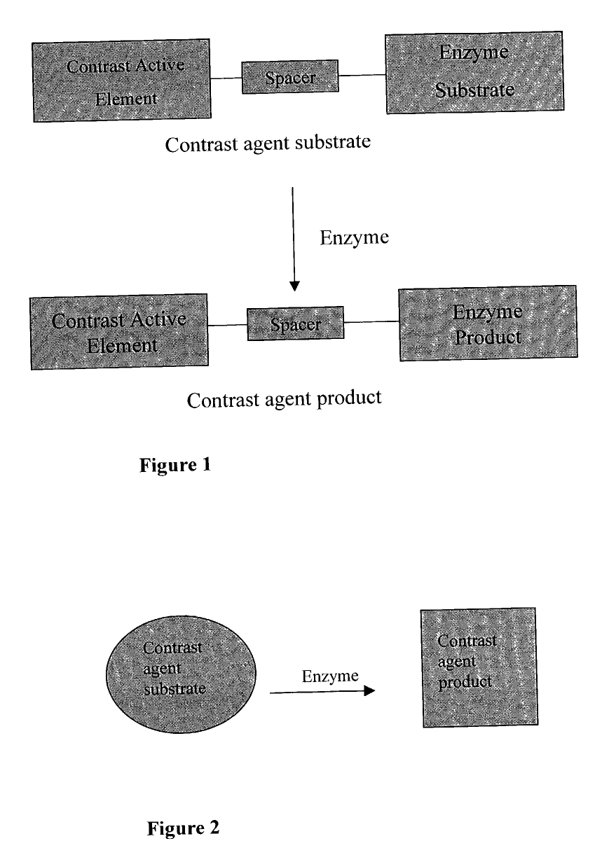

The novel contrast agents are substrates for one or more enzyme(s) and the result of enzyme activity results in contrast agent products that either have different contrast efficacy than the contrast substrate and/or have different pharmacokinetic and/or pharmacodynamic properties than the contrast substrate. FIGS. 1 and 2 schematically show the transformation of a contrast agent substrate to a contrast agent product under influence of enzymes.

Several in vivo methods, both imaging techniques and non-imaging techniques, can be used to diagnose disease. Typical non-imaging techniques include simple blood pressure measurements, electrocardiography and electrocephalography for detection of electric currents in the heart muscle and brain, respectively, and other simple test performed in doctors offices/hospitals for diagnosis of disease. Much more information, including spatial information, is obtained by the use of imaging techniques. The most frequently used methods include various X-ray based techniques, MRI, ultrasound and diagnostic methods based on radioactive materials. Other diagnostic imaging methods include optical imaging modalities, Overhauser MR (OMRI), oxygen imaging (OXI) which is based on OMRI, magnetic source imaging (MSI), applied potential tomography (APT) and potential imaging methods based on microwaves.

The images obtained in X-ray techniques reflect the different densities of structures/organs/tissue in the patients body. Contrast agents are today used to improve the image contrast in soft tissue examinations. Examples of such contrast agents include gas (negative contrast effects relative to tissue), barium sulphate suspensions and iodinated agents; ionic monomeric agents, non-ionic monomers, ionic dimers and non-ionic dimers. Typical commercial X-ray contrast agents are Omnipaque® and Visipaque®.

MRI imaging is an imaging method based on interaction between radio waves and body tissue water protons in a magnetic field. The contrast parameter or signal intensity is dependent on several factors including proton density, spin lattice (T₁) and spin spin (T₂) relaxation times of water protons.

Ultrasound is another valuable modality in diagnostic imaging that does not use ionizing radiation. In ultrasound examinations the patient is exposed to sound waves in the frequency range of 1-10 MHz. These sound waves (or ultrasound waves) penetrate through tissue or are reflected from the tissue. The reflection of these sounds is detected by a transducer and form the basis for development of an ultrasound image. Ultrasound imaging is a method of choice in pregnancy checks and birth control and diagnosis of cardiovascular diseases and liver diseases.

All practical ultrasound contrast agents are based on encapsulated gas because the reflection of sound from the liquid-gas interface is extremely efficient. Typical ultrasound contrast agents are gas encapsulated in a sugar matrix, in a shell of denaturated albumin/or partly denaturated albumin, in polymers or in surfactants including phospholipids. A typical ultrasound contrast agent with high contrast efficacy consists of a perfluorocarbon gas bubble (for example perfluoropropane or perfluorobutane) coated with a layer/layers of phospholipids. The particle size is around 4 micrometer with very few particles larger than 10 micrometer in diameter. The main indications for such a typical product will in the future be cardiac imaging (cardiac perfusion examinations) and liver imaging.

Nuclear medicine imaging modalities are based upon administration of radioactive isotopes followed by detection of the isotopes using a gamma camera or positron emission tomography (PET). The most frequently used examination is gamma camera detection of 99m-technetium in the form of a chelate; for example, a technetium phosphonate chelate for bone scintigraphy.

Optical imaging methods are performed using contrast agents that absorb light (e.g. near infrared light) with or without subsequent re-emission (fluorescence/phosphorescence).

MSI methods are performed without contrast agent (detection of natural magnetic fields in the human body), however, contrast agent based on magnetic materials may improve this technique substantially.

APT based methods can also be performed (as for instance, thallium scans) without use of contrast agents, however, contrast agents based on physiologically acceptable ions or other agents with effect on conductivity improve the diagnostic utility of APT.

All these different modalities complement each other with regard to diagnosis based on morphology/anatomy. However, none of these modalities as used today are useful for diagnosis based on physiological parameters other than studies of function, for instance blood perfusion (blood supply), blood flow and active cell uptake (thallium scans in cardiac stress a.s.o.).

In the past there has been a great interest for measurements and quantification of various physiological parameters, however very few of the methods used involve administration of contrast agents and direct observation of the effect of the contrast agent in a 2D- or 3D image. Most of these methods measure or try to measure physiological parameters like temperature, pH, oxygen tension and calcium.

WO 99/51994 (Jenkins et al) describes a method using MRI for detecting changes in neurotransmitter and neuroreseptor activity as a metabolic response to diagnostic challenge or therapeutics in psychiatric patients with suspected or already diagnosed mental illness. This application does not relate to enzyme activity.

Calvo et al in Surg. Oncol. Clin. North. Am., 1999, 8, 171-183 deals with MR imaging as a molecular diagnostic tool beyond anatomy and the discussion includes MR imaging of gene expression. According to Caravan et al in Chem. Rev. 1999, 99, 2293-2352, and WO 97/36619 (Lauffer et al) a contrast agent precursor changes binding properties to a protein upon an enzymatic transformation to a contrast agent. Proteins are e.g. plasma proteins or proteins present in other body fluids.

WO 99/17809 (Lauffer et al) claims contrast agents comprising an image-enhancing moiety and a state-dependent tissue binding moiety. Lauffer et al focus on the monitoring of interventional therapy and does not relate imaging to enzyme activity.

Agents for mapping of metabolic processes or more specifically enzyme activity are sparsely described in the literature; Weissleder et al have recently described protease-activated near infrared probes for tumor imaging. In Nature Biotechnology 1999, 17, 375-378, Weissleder has further in an editorial article, Radiology 1999, 212, 609-614, discussed “molecular imaging:exploring the next frontier”. In this publication the only enzyme target for MR contrasts is β-galactosidase.

Moats RA et al in Angew Chem Int Ed Engl. 1997, 36, 726-728 have described an MR contrast agent substrate for β-galactosidase. The enzyme activated cleavage of the galactopyranose and the change of coordination number of gadolinium results in a relative small change in relaxivity. When relaxivity changes are of this magnitude, the local concentration of contrast agent in normal tissue and pathological tissue has to be the same (or has to be quantified) to have reliable diagnostic results based on differences in enzyme activity.

U.S. Pat. No. 5,707,605, U.S. Pat. No. 5,980,862, WO 96/38184, WO 99/25389 (Meade et al) describe MR contrast agents comprising a complex consisting of a paramagnetic metal ion and a chelator comprising a moiety covalently attached to the chelator that occupies a coordination site and that may be removed by enzymatic cleavage of a bond in said moiety. A drawback with the disclosed invention is that the change in relaxivity caused by the enzymatic activated transformation is relatively small. Inherent differences in concentration may overrule the effect of relaxation changes caused by the enzymatic transformation of the contrast agent. In contrary to the contrast agents disclosed by Meade et al, the contrast agents according to the present invention do not have a blocking agent that is cleaved off.

WO 99/58161 (Weissleder et al) claims an intramolecularly quenched fluorescence probe comprising a polymeric backbone and a plurality of near infrared fluorochromes covalently linked to the backbone at fluorescence-quenching interaction - permissive positions separable by enzymatic cleavage at fluorescence activation sites.

WO 98/33809 (Bogdanow et al) suggests composition and methods for imaging gene expression.

Anelli et al in Eur. J. Inorg. Chem. 2000, 625-630 describes a gadolinium chelate targeting the enzyme carbonic anhydrase. The chelate is not a substrate for the enzyme. The usefulness of isotope-labelled molecules like ¹⁸F-labelled glucose ([¹⁸F]FDG) in PET imaging has come in focus during the last 10 years. The focus on [¹⁸F]FDG is related to its properties as a multifunctional radiopharmaceutical, since it can be used for mapping of enhanced glucose metabolism. The metabolism of glucose and [¹⁸F]FDG is very similar during the first steps, giving intracellular uptake and generation of 2-deoxy-2-[¹⁸F]fluoro-D-glucose-6-phosphate. However, the latter is not a substrate for the next step in the metabolic pathway, dephosphorylation by glucose-6-phosphatase, because of the blocking effect of the fluorine atom in the 2-position. This results in intracellular trapping of contrast in the form of 2-deoxy-2- [¹⁸F]fluoro-D-glucose-6-phosphate, see e.g. B. Beuthien-Baumann et al, Carbohydrate Res. 327 (2000) 107-118 or A. Saleem et al, Advanced Drug Delivery Reviews 41 (2000) 21-39. Thus, since the glucose metabolism in e.g. tumours is enhanced, [¹⁸F]FDG can be used to visualise different forms of cancer. Even if FDG is transformed to glucose fluoro-deoxy-6-phosphate the contrast mechanism is not based on a change in pharmacodynamic or pharmacokinetic properties. Rather the contrast mechanism is simply based upon that FGD is actively transported over a cell membrane and into a cell with a high metabolism. However, the current invention does not relate to contrast agents being detected based on a transport mechanism by itself or a simple targeting mechanism. Rather it requires a change upon the pharmacodynamic or pharmacokinetic properties between a contrast agent substrate and a contrast agent product.

Pinnaduwage et al Clin. Chem. 34/2, (1988) 268-272 has described stable liposomes with entrapped glucose-6-phosphate dehydrogenase prepared with unsaturated phosphatidylethanolamine stabilised with ganglioside GM₁. Addition of β-galactosidase caused rapid lysis of liposomes. Pinnaduwage does not disclose contrast agents.

However, there is still a need for contrast agents that can enable diagnosis of diseases in an early stage with good reliability. We have surprisingly discovered that using contrast agents that change pharmocodynamic or pharmacokinetic properties in a chemical modification activated by an enzyme or enzyme systems fulfill these requirements.

The invention is also described in the claims.

The follwing definitions will be used throughout the document:

Contrast agent: Molecular moiety used for enhancement of image contrast in vivo comprising at least one contrast active element. The contrast agent may in addition comprise an enzyme substrate.

Contrast agent substrate: A contrast agent comprising at least one enzyme substrate and at least one contrast active element.

Enzyme substrate: Molecular moiety on which an enzyme acts. When the enzyme substrate is part of a larger molecular moiety the enzyme substrate defines the part of the moiety wherein a chemical modification occurs.

Contrast active element: Molecular moiety giving an enhanced image contrast in diagnostic imaging.

Contrast agent product: A product from a contrast agent substrate having been processed by one or more enzymes.

The present invention provides a contrast agent substrate susceptible of changing pharmacodynamic and/or pharmacokinetic properties upon the influence of enzymatic activity.

Hence, the present invention provides contrast agent substrates that change pharmacodynamic and/or pharmacokinetic properties upon a chemical modification from the contrast agent substrate to a contrast agent product in a specific enzymatic transformation, and thereby enablingdetectiion of areas of disease upon a deviation in the enzyme activity from the normal.

One aspect of the present invention provides contrast agents for identification and /or diagnosis of tissue or cells with abnormal metabolic activity (increased or decreased), wherein the contrast agent comprises a contrast agent substrate that changes pharmacodynamic and/or pharmacokinetic properties upon a chemical modification from a contrast agent substrate to a contrast agent product upon a specific enzymatic transformation.

Another aspect of the invention is contrast agent substrates for identification/diagnosis of cancer, cardiovascular diseases or inflammations or infections, wherein the contrast agent substrate changes pharmacodynamic and/or pharmacokinetic properties upon a chemical modification from a contrast agent substrate to a contrast agent product upon a specific enzymatic transformation and wherein abnormal enzyme activity is shown at an area of disease.

The contrast agents may be produced and used for diagnosis of diseases in humans and animals based on mapping of a change in metabolic activity. The new contrast agents are substrates for enzymes. The contrast media substrates are transformed into contrast agent products in a reaction activated by enzyme(s), and detected in an imaging technique.

One aspect of the present invention is contrast agent substrates that change pharmacodynamic properties as a result of the metabolic activity. According to one of the aspects of the present invention, the contrast agents change efficacy as a result of changes in pharmacodynamic properties, e.g. an MR contrast medium that changes binding properties to biological components.

Another aspect of the present invention is contrast agent substrates that change pharmacokinetic properties upon enzymatic modifications.

One embodiment of the present invention is contrast agent substrates for diagnosis of cancer or cancer-related disease based on mapping of changes in metabolic activity/enzyme activity.

Another embodiment of the present invention is contrast agent substrates for diagnosis of diseases related to the cardiovascular system based on mapping of changes in metabolic/enzyme activity.

Yet another embodiment of the present invention is metabolic/enzyme specific contrast agent substrates for diagnosis of inflammations and infections.

Still another embodiment of the present invention relates to diagnose of diseases on the central nervous system based on changes in metabolic activity.

Contrast agents for diagnosis of cancer or cancer-related disease based on mapping of changes in metabolic activity/enzyme activity is a preferred embodiment of the application.

A preferred embodiment of the invention is a MR contrast agent substrate or a scintigraphic contrast agent substrate that changes pharmacodynamic and/or pharmacokinetic properties upon a chemical modification of the contrast agent substrate to a contrast agent product upon a change in enzyme activity in a specific enzymatic transformation. A MR contrast agent is specifically preferred.

Contrast agents according to the invention are substrates for specific enzymes. These contrast agent substrates are transformed into contrast agent products through a chemical modification activated by at least one enzyme, i.e. one enzyme or an enzyme system. The contrast agents can be used in identification of tissue/cells with reduced metabolic or enzymatic activity or preferably to identification of tissue or cells with increased metabolic or enzymatic activity compared to healthy tissue/cells.

One aspect of the invention is a method for identification/diagnosis of tissue or cells with abnormal metabolic activity using a contrast agent substrate that changes pharmacodynamic and/or pharmacokinetic properties upon a chemical modification of the contrast agent substrate to a contrast agent product upon a specific enzymatic transformation.

Another aspect of the invention is a method for identification/diagnosis of cancer, cardiovascular diseases or inflammations or infections using a contrast agent substrate that changes pharmacodynamic and/or pharmacokinetic properties upon a chemical modification of the contrast agent substrate to a contrast agent product upon a specific enzymatic transformation wherein abnormal enzyme activity is shown at an area of disease.

Yet another aspect of the present invention is contrast agent substrates that change pharmacodynamic properties as a result of metabolic transformation. The change in pharmacodynamic properties may be that the metabolic transformation results in changes in specific or non-specific binding properties of the contrast agent substrate to biological surfaces, e.g.:

changes in receptor affinity, and/or

changes in cell surface binding properties, and/or

changes in intracellulare binding to macromolecules, and/or

changes in binding/affinity for any endogenous compound or biological structure

changes in intracellular accumulation or concentration

Preferably the receptor affinity is changed by a factor of at least 0.5 and more preferably by a factor of at least 1 and most preferably by a factor of at least 3, and/or the non-specific cell surface binding properties is changed by a factor of at least 0.5 and more preferably by a factor of at least 1 and most preferably by a factor of at least 3, and/or the binding to intracellulare macromolecules is changed by a factor of at least 0.5 and more preferably by a factor of at least 1 and most preferably by a factor of at least 4, and/or the change in binding/affinity for endogenous compounds or biological structures is changed by a fator of at least 0.5 and more preferable by a factor of at least 1 and most preferably by a factor of at least 3, and/or the intracellular accumulation or concentration is changed by a factor of at least 0.5 and more preferably by a factor of at least 1 and most preferably by a factor at at least 3.

Another aspect of the present invention is contrast agent substrates that significantly change pharmacokinetic properties as a result of the metabolic transformation. The changes in pharmacokinetic properties can be that the metabolic transformation results in:

changes in plasma clearance, and/or

changes in renal clearance, and/or

changes in hepatic clearance, and/or

changes in rate of penetration of biological membranes, and/or

changes in membrane permeability or affinity for a transport protein and/or

changes in volume of distribution

Preferably the plasma clearance is changed by a factor of at least 0.5 and more preferably by a factor of at least 1 and most preferably by a factor of at least 3, and/or the changes in renal clearance is changed by a factor of at least 0.5 and more preferably by a factor of at least 1 and most preferably by a factor of at least 3 , and/or changes in hepatic clearance is, changed by a factor of at least 0.5 and more preferably by a factor of at least 1 and most preferably by a factor of at least 3, and/or changes in penetration rate of biological membranes is changed by a factor of at least 0.5 and more preferably by a factor of at least 1 and most preferably by a factor of at least 3e, and/or the change in volume of distribution is changed by a factor of at least 0.5 and more preferably by a factor of at least 1 and most preferably by a factor of at least 2.

According to another aspect of the present invention, the contrast agent products have different efficacy, especially r₁ relaxivity in MRI, than the contrast agent substrates. The preferred change in relaxitiy is preferably at least 30% and the ratio of relaxivity between contrast media product and contrast agent substrate is most preferably at least 2 or less than 0.5. The coordination number of the paramagnetic chelate is the same before and after the enzyme-activated transformation. The change in relaxivity may e.g. be a result of different tumbling rate for the contrast agent substrate and the contrast agent product based on different affinity for biological surfaces or macromolecules.

The transformation of a contrast agent substrate to a contrast agent product upon an enzymatic activity preferably gives a change in the pharmacodynamic properties. Preferably the metabolic transformation results in a change in receptor affinity, e.g. meaning that the contrast agent product has a stronger bond to the receptor, e.g. cell surface or to macromolecules, than the corresponding contrast agent substrate has.

Contrast agents having a targeting effect are well known. A probable drawback with a targeting contrast agent comprising an enzyme substrate could be the rapid turnover rates of the enzymes substrates. This means that the residence time for a substrate in the active site of the enzyme on the target tissue is short, as the cleavage products, i.e. contrast active elements, are washed away in the blood stream. A solution to this problem has been sought.

Hence, a further embodiment of the invention is a contrast agent substrate as earlier described further comprising a targeting vector.

The contrast agent substrate according to this embodiment of the invention would hence be a contrast agent substrate for detecting enzyme activity characterized in that the contrast agent substrate changes pharmacodynamic properties and/or pharmacokinetic properties upon a chemical modification of the contrast agent substrate to a contrast agent product upon a specific enzymatic transformation and wherein the contrast agent substrate comprises a contrast active element, a targeting vector and an enzyme substrate.

The following mechanism for an increased residence time is suggested:

a) The enzyme substrate is processed by the enzyme

b) The enzyme substrate liberates the contrast active element attached to the targeting vector

c) The targeting vector attached to the contrast active element is bound to a target/receptor in or around the diseased area

As a result the contrast active element is retained in or around the diseased area and thus enhancing binding/uptake of the contrast active element and hence the residence time.

The order of the steps of the mechanism may also be changed. In addition, the enzyme substrate preferably binds to the disease specific enzyme before it is processed by the enzyme.

A contrast agent substrate according to this aspect of the invention would preferably be a contrast agent for MR or nuclear medicine, typically a gadolinium or technetium chelate. The enzyme substrate could be any substrate for a disease specific enzyme or enzyme system, but would preferably be a substrate for a tumour specific enzyme such as those given in list 1. Substrates for AMP-N and Cathepsin D are specifically preferred.

The targeting vector could be any targeting vector known from the literature, but vectors having affinity for tumour specific receptors are preferred.

The process referred to in step b) could be any chemical modification referred to in this document, but preferably the process involves a cleaving of the bond between the targeting vector and the enzyme substrate.

More specifically, a contrast agent according to this aspect of the invention could solve the problem with low residence time by combining enzyme targeting/cleavage with a pH dependent switch for proteoglycan targeting. This field of the invention is directed to identification and/or diagnosis of cancer.

Proteoglycans are high molecular weight polyionic substances comprising mainly glycosaminoglycan chains linked covalently to a protein core. The high charge and structure of these macromolecules is essential for their role as key supporting elements in connective tissue and extracellular matrix. During growth and metastasis the tumour cells produce a range of enzymes which help breakdown and modify the architecture of the surrounding tissue. At this stage in a tumour's development the proteoglycans are both available for targeting by blood born agents and more crucially, experience a lower pH environment than in normal health tissue.

A class of glycoprotein known as His-pro-rich glycoproteins have an affinity for glycosaminoglycans which is pH dependent. The ionic charge is exquisitely sensitive to pH in the range 5.5-7 with binding following protonation of multiple histidine residues. As most tumour tissues have a low pH in comparison to healthy tissue this mechanisme can be used for tumour targeting.

For tumour targeting the contrast agent substrate preferably comprises:

1) An enzyme substrate for a tumour specific enzyme

2) A peptide sequence

3) A contrast active element

The enzyme substrate is preferably AMP-N or Cathepsin D. The peptide sequence could be any peptide sequence which is non-protonated at physiological pH but when retained at the tumour site, where the pH is lower, becomes protonated and binds proteoglycans following enzymatic cleavage. The peptide sequence is preferably a series of (GHHPH)n peptide sequences wherein n is a number between 1 and 20, and more preferably between 5 and 15. The peptide sequence could also be exchanged with any moiety giving a cationic charge at the contrast active element part of the moiety after cleavage. The contrast active element is preferably a chelate or moiety suitable for introduction of radionuclide or metal for MR imaging.

Binding of the enzyme substrate to the tumour bound enzyme causes a brief retention of the agent before cleavage. This will typically have fast kinetics. Although the cleavage reaction of the binding between the substrate enzyme and peptide happens quickly the His-Pro sequence switches to protonated form during this step due to the low pH at the binding site. Cleavage occurs liberating the now free protonated His-Pro rich peptide which is further retained by binding to proteoglycans in or around the tumour. This last step typically has slow kinetics, giving an increased uptake, retention and tumour to background contrast.

Contrast agents according to this part of the invention are suggested in examples 1 and 2, for MR and nuclear imaging, respectively.

The target enzymes for these contrast agents vary with the indication. The target enzymes are usually enzymes that exhibit altered activity in the diseased area. The activity is usually increased, but it may also be decreased at the diseased area compared to other areas. Increased activity may usually be assumed to result from overexpression of the gene, but other mechanisms may also influence the activity. Lists 1 to 5 list some enzymes associated with cancer, cardiovascular diseases, the central nervous system (CNS), bone diseases and infections. Enzymes associated with cancer are generally described by Sakurai, Y. et al in Surg. Today, 1998, 28, 247-57.

### LIST 1

### Examples Of Some Enzymes Associated With Cancer

Alkaline phosphatase, Aromatase, N-acetylglucosaminyltransferase, 17-alpha-hydroxylase/17,20-lysae (CYP17), Cathepsin D, Cyclooxygenase, Cysteine protease, Dihydropyrimidine dehydrogenase (DPD), Famesyltransferase, Fucosyltransferase, Glutamyl hydrolase, Glutathione S-transferase, Glycogen phosphorylase (GP), Lipoxygenase, 12-Lipoxygenase, Matrix metalloproteinase, Nitric oxide synthetase, Oestradiol 17β-hydroxy steroid dehydrogenase, Proteolytic enzymes in general, Phosphatases, Phospholipase C, Phosphodiesterase (PDE 1), Phospholipid phosphatase, Protein kinase C, Pyruvate kinase, Ribonucleases (acid RNases), Steroid sulphatase, Stearoyl -CoA desaturase, Testosterone 5-alpha-reductase, Thymidyl synthetase, Topoisomerase, Telomerase, Tyrosine kinase.

### LIST 2

### Examples Of Some Enzymes Associated With Cardiovascular Diseases

Angiotensin—Converting enzyme (ACE), Ca(2+)—Transporting ATPase, Hydroxymethylglutaryl-CoA reductase, Cyclic AMP-dependant protein kinase, Endopeptidases, Endothelial constitutive nitric oxide synthase, Inducible nitric oxide synthase, Nitric oxide synthase, Cyclooxygenase 2, Prostaglandin endoperoxide synthase, Aspartic endopeptidase, Endothelin converting enzyme, Beta-adrenergic receptor kinase, G-protein-coupled receptor kinase-3, G-protein-coupled receptor kinase-5, Protein-Serine-Threonine kinase, Peptidyl-dipeptidase A, 3′,5′-cyclic-GMP phosphodiesterase, Protein kinase C, Esterase, Aryldialkylphosphatase, Creatine kinase, Dopamine beta-hydroxylase, Fatty acid desaturase, Serine endopeptidase, Phosphoprotein phosphatase, Acetyl CoA carboxylase, Cystathionine beta-synthase, Methylenetetrahydrofolate reductase, Superoxide dismutase, Paraoxonase, Thrombin,Plasmin, Factor VIIa, Factor Ixa, Factor Xa, Streptokinase, Urokinase, Plasminogen Activator.

### LIST 3

### Examples Of Some Enzymes Associated With The Central Nervous System (CNS)

Protein kinases, Phosphopyruvate hydratase, Ca(2+)-transporting ATPase, Amonihydrolases, Aspartocyclase, Nitric oxide synthase, Choline O-acetyltransferase, Monoamine oxydase, beta-1,4-galactosyl transferase, Myelin basic protein kinase, Cyclooxygenase-2, Endothelial constitutive nitric oxide synthase, Amino-acid neurotransmitters, Phosphoprotein phosphatase, Alkaline phosphatase, Nucleotidase, Catechol O-methyltransferase, Glutamyl carboxylase, Glutamate translocase, Glutamate decarboxylase, Acetylcholinesterase, Tyrosine 3-monooxygenase, Peptide hydrolases, Aminopeptidase, Hydrolases.

### LIST 3a

### Examples Of Some Enzymes Of Specific Importance For Alzheimer Disease

Choline O-acetyl-transferase, Cyclooxygenase-2, Matrix metalloproteinase, Protease, Nitric-oxide synthase, Phospholipase A2, Acetylcholinesterase, Calpain, Endopeptidases.

### LIST 3b

### Examples Of Specific Enzymes Related To Multiple Sclerosis (MS)

Matrix metalloproteinase, Phosphodiesterase 4, Nitric oxide synthase, Gelatinase B.

### LIST 4

### Examples Of Specific Enzymes Related To Bone Diseases

Alkaline phosphatase, Acid phosphatase, Tartrate-resistant acid phosphatase, Metalloendopeptidase, Collagenases, Nitric-oxide synthase, Aromatase.

### LIST 5a

### Examples Of Some Viral Enzymes Associated With Virus Infections

Alpha-glocosidase, RNA Repliase, Endopeptidase, Cystein endopeptidase, DNA helicase, Herpes simplex thymidine kinase,(HSV-TK), Serine endopeptidase,Influenza A and B viral neuramidase, Hepatitis C virus helicase, Viral NS3 serine, Protease, RNA helicase, RNA dependent RNA polymerase, Ribonucleotide reductase, Viral protease, Viral kinase, HIV reverse transcriptase, Viral integrase, RNA-directed DNA polymerase, Alanine transaminase.

### LIST 5b

### Examples Of Some Enzymes Associated With Bacterial Infections

beta-lactamase, Carbohydrate dehydrogenase, Aryl and alkyl transferase, Peptide synthease, Serine endopeptidase, Topoisomerase, Muramidase, Acetyltransferase, Phosphotransferase, MASP-2 protease, MBP-associated serine protease, Amidohydrolase.

### LIST 5c

### Examples Of Some Enzymes Associated With Fungal Infections

TOR kinase, 1,3-beta-glucan synthase, Lysophospholipase, Calcineurin, Chitin synthetase, Phospholipase, Beta-N-acetylhexoaminidase, H⊕-ATPase, Glycylpeptide-N-myristoyl transferase, Methyltransferase The contrast agents according to this invention can in principle be used in conjunction with any diagnostic imaging modality. It is preferred that contrast agents according to the invention is used for imaging of the human body based on magnetic resonance imaging, ultrasound, optical imaging, nuclear medicine techniques or x-ray. The most preferred imaging modalities are MRI and nuclear medicine based techniques. MRI is specifically preferred.

The MR contrast active element of the contrast agent substrate according to the invention is a paramagnetic compound, a magnetic (super-paramagnetic) compound, a ferrimagnetic or ferromagnetic compound, and/or a fluorinated compound. The MR contrast active element according to the invention can also comprise hyperpolarized compounds, e.g. NMR active nucleus such as ¹³C, ¹⁵N, ¹⁹F, ³¹P, ¹H, ²⁹Si. The most preferred contrast active elements are paramagnetic chelates and iron based super-paramagnetic compounds. Preferred paramagnetic chelates include chelates of transition metals or lanthanide metals, e.g. manganese, gadolinium, ytterbium and dysprosium. The most preferred paramagnetic element is gadolinium.

Preferred magnetic (super-paramagnetic) compounds include uncoated and coated particles of magnetic iron oxide and (-iron oxide and other iron/metal oxides with high magnetic susceptibility. Preferred fluorinated compounds are compounds with relatively short ¹⁹F T₁-relaxation times. Other preferred fluorinated compounds according to the present invention are fluorinated pH-probes. An MR contrast agent substrate according to the invention would typically comprise any of the above mentioned contrast active elements and being a contrast agent substrate for enzymatic metabolic transformation. The contrast agent substrate can for example comprise a contrast active element coupled to a specific enzyme substrate optionally through a spacer.

The ultrasound contrast active ingredient according to the present invention will generally be a gas-containing bubble or a precursor of such gas bubble. Any biocompatible gas may be employed in the contrast agents of the invention including any substances (including mixtures) that is substantially or completely in gaseous (including vapour) form at the normal human body temperature of 37EC. Preferred gases are halogenated hydrocarbon gases, especially fluorinated hydrocarbon gases, e.g. perfluorobutane. A list of gases that can be used is given in WO 9729782 and is included here by reference. In vesicles filled with gas, the membrane may be formed of any physiologically tolerable membrane forming material, in particular phospholipids, and may be cross-linked or non-cross-linked. Membranes formed of mixtures of charged and non-charged phospholipids are especially preferred and it is particularly preferred that the vesicles should carry a net surface charge, preferably a negative charge. Different membrane forming materials are given in WO 9729783 and are included here by reference. Ultrasound contrast agent substrates according to the invention may be divided into two main groups: Those which experience a change in encapsulation material giving a change in physical/chemical properties (e.g. change of size or stability) upon enzymatic influence, and those which experience a change in surface properties, e.g. the binding properties upon enzymatic influence. The contrast agent substrates may be microbubbles that are susceptible to enzymatic modifications in vivo and preferably have at least partly wall material consisting of molecules that are substrates for disease related enzymes. The products of the enzymatic reactions will preferably differ from the substrates with regard to electric charge, hydrofobicity, exposed ligands etc. Reactions from contrast agent substrates to contrast agent products wherein the reactions involve hydrolysis of peptides in the membrane are described later in this document.

The microbubbles may be made with exposed amino groups. Microbubble substrates for hyaluronidase may for instance comprise hyaluronic acid (a high-molecular weight copolymer of glucuronic acid and N-acetyl-glucosamine) coupled to the microbubbles at a limited number of points, for instance, by a water-soluble cardodiimide. Hyaluronidase will hydrolyse the hyaluronic acid, leaving essentially oligosaccharides that are bound by amide bonds. The surface properties of the microbubble will now revert to its original state.

It may be desirable to have a net charge on the product microbubble. This may be achieved by including a non-modifiable charged compound; e.g., a microbubble containing stearylamine and palmityl phosphate in a ratio of 1:2 would be negatively charged. After the phosphate groups were removed by alkaline phosphatase, the positive charges of the stearylamine would be left.

For the detection of proteases, N-blocked peptide (e.g., N-acetyl-) substrates could be attached to the microspheres. Cleavage of the peptide would leave a positive charge on the resulting amino terminus. Alternatively, the peptide substrate could be attached by its amino terminus, and the carboxy terminus esterified or amidated. In this instance, cleavage would increase the negative charge by 1 unit.

Ligands for cell-surface receptors may be made into enzyme substrates by chemical modifications that simultaneously block receptor binding. For instance, terminal (non-reducing) galactose residues that are esterified on one hydroxyl group are not recognized by the hepatic asialo-glycoprotein receptor, but the phosphate group may be removed by phosphatases.

The substrates described above would work equally well for changing the properties of liposomes, either for diagnostics or drug delivery. Enzyme sensitive liposomal contrast agents, for exampel for MR, according to the invention may also change their surface properties as a result of an enzymatic transformation. Paramagnetic amphiphilic liposomes comprising an enzyme cleavable bond in the headgroup may be produced. The headgroup may be prepared such that a cleavage of the enzyme cleavable bond would cause a break down of the liposomes to non-lamellar structures and subsequent release of the encapsulated contrast agent to the surroundings. The new structures after the enzymatic transformation will be dependent on the size of the headgroup after the cleavage.

Radiopharmaceutical contrast agent active elements according to the present invention are radioactively labeled compounds comprising isotopes useful for imaging. These compounds can have the radioisotope covalently bound (e.g. 18-F and ¹¹C) or in the form of a chelate (e.g. Technetium). The contrast generating species in nuclear medicine contrast agents for use according to the invention may be any radioactive compound of the type in diagnostic nuclear medicine, for example known compounds useful for scintigraphy, SPECT and PET. Typical compounds include

radioiodinated compounds, ¹¹¹Indium labelled materials and ^{99m}Tc labelled compounds (for example ^{99m}TcDTPA, ^{99m}TcHIDA and ^{99m}Tc labelled polyphophonates) and ⁵¹CrEDTA. Contrast agent substrates for nuclear medicine would typically comprise a radiopharmaceutical contrast agent as described above and being a contrast agent substrate for enzymatic metabolic transformation. The contrast agent substrate can for example comprise a contrast active element coupled to a specific enzyme substrate optionally through a spacer.

Optical imaging contrast agents according to the invention would typically comprise a contrast active element that either absorbs light energy, with or without subsequent re-emission at a lower energy (fluorescence, phosphorescence), or scatters the incident photons. Optical probes have the ability to change their optical properties as a result of their local physical and/or chemical environment. More specifically, enzymatic activity may alter these optical properties.

An optical imaging contrast agent substrate of the invention may comprise a fluorescent dye (fluorophore, fluorochrome, fluorescent probe) that may be quenched (no fluorescence occurs) by associated quencher groups, until an enzymatic cleavage that separates the dye from the quencher de-quenches the dye with induced fluorescence as a result.

In one embodiment of the invention the contrast agent substrate comprises a fluorescent dye which may have its fluorescence characteristics altered by enzymatic activity, such as its Stokes shift, quantum yield or lifetime/decay kinetics.

In another embodiment, an absorbing (non-fluorescent) dye may be altered by enzymatic activity in such a way that the absorption spectrum is shifted (change in ‘color’).

In yet another embodiment the contrast agent substrate is a particle that due to its size will scatter the incident photons, and that may be altered by enzymatic activity in such a way that the size is increased or reduced, thereby changing its ability to scatter photons at the incident wavelength(s). Such changes may be due to dissolution (disappearance), swelling or shrinkage.

One of the main clinical advantages with contrast agents according to this invention is that these metabolically sensitive contrast agents are more sensitive to pathology than morphological contrast agents. As abnormal enzymatic activity is an early sign of a disease/condition the new contrast agents have a potential for diagnosing diseases at an early stage, which in many clinical situations are very important for the outcome of the treatment. Another clinical advantage using these new contrast agents relative to state of art agents is that they are very sensitive to treatment and therefore can be used to follow up treatment. Early diagnosis and the possibility for follow up therapy are clinically important in many diseases including cancer diagnosis and therapy.

A preferred embodiment of the present invention relates to new contrast agents for diagnosis of cancer and cancer related diseases based on mapping of metabolic activity/enzyme activity in the tissue. Contrast agents according to this invention for diagnose of cancer can target any enzyme relevant for cancer. Preferred enzyme targets for diagnosis of cancer are listed in List. 1. The most preferred enzyme targets for diagnosis of cancer are cyclooxygenase, farnesyltransferase, matrix metalloproteinases, topoismerase and telomerase.

Another embodiment of the present invention relates to new contrast agents for diagnosis of cardiovascular diseases based on mapping of enzyme activity. The new contrast agents will be useful in diagnosis of cardiac failure, myocardial infarction, atherosclerosis, thrombosis, embolism, aneurysms, stroke, and hemorrhage. Preferred diseases are atherosclerosis, myocardial infarction and thrombosis. Preferred enzyme targets are listed in List 2. The most preferred enzyme targets for diagnosis of cardiovascular diseases are angiotensin—converting enzyme (ACE), hydroxymethylglutaryl-CoA reductase, endothelial constitutive nitric oxide synthase, inducible nitric oxide synthase, nitric oxide synthase, endothelin converting enzyme, protein serine-threonine kinase, phosphoprotein phosphatase, superoxide dismutase, thrombin, plasmin, plasminogen activator and lipoprotein lipase.

Another embodiment of the present invention relates to new contrast agents for diagnosis of diseases in the central nervous system (CNS). Preferred enzyme targets are listed in List 3. Most preferred enzyme targets are protein kinases, nitric oxide synthase, monoamine oxydase, myelin basic protein kinase, phosphoprotein phosphatase, glutamate translocase, tyrosine 3-monooxygenase, hydrolases.

One preferred disease in CNS is Alzheimer's disease. Preferred enzyme targets for diagnosis of Alzheimer's disease are listed in List 3a. Most preferred enzyme targets are matrix metalloproteinase, protease and calpain. Another preferred disease in CNS is multiple sclerosis (MS). Preferred enzyme targets for diagnosis of MS are listed in List 3b. The most preferred enzyme target is matrix metalloproteinase.

Another embodiment of the present invention relates to new contrast agents for diagnosis of bone diseases. Preferred diseases are osteolytic diseases, for instance osteoporosis, and osteopetrosis and osteosclerosis. Preferred enzymes targets for diagnosis of such diseases are listed in List 4. Most preferred enzyme targets are alkaline phosphatase, acid phosphatase and collagenases.

Still another specific embodiment of the present inventions relates to diagnosis of infections. Preferred enzyme targets for diagnosis of infections are listed in List 5a (virus infections), List 5b (bacterial infections) and List 5c (fungal infections). Most preferred enzyme targets for viral infections are RNA replicase, endopeptidase, DNA helicase, viral neuramidase, [HIV] reverse transcriptase, viral integrase and proteases. Most preferred enzyme targets for bacterial infections are beta-lactamase, serine endopeptidase, muramidase. Most preferred enzyme targets for fungal infections are 1,3-beta-glucan synthase, calcineurin, chitin synthetase, glycylpeptide-N-myristoyl transferase.

Certain contrast agent substrates may also be used in identification of apoptosis and necrosis. Hence, new contrast agents may be used in diagnosis of diseases based on identification of apoptosis and necrosis. Apoptosis is the internal programmed process of cell death inactivating the genetic material and crucial parts of the metabolic machinery. Necrosis is the pathological process of destruction of tissue due to external insults, although there is no dividing line between apoptosis and necrosis.

In mature individuals, aptoptosis of large numbers of cells within a small volume of tissue will frequently be a sign of disease, while apoptosis of single cells (for instance, senescent granulocytes) occurs continuously. Apoptosis is initiated by signals which may either be external (i.e., tumour necrosis factor-∀ or Fas ligand) or internal. Internal signals may be generated by failure of repair mechanisms for DNA damage (e.g., p53), loss of adhesion to the substrate, or stress factors such as low pH, low energy supply, or UV light. The process proceeds through several distinctive steps, including loss of mitochondrial membrane potential, release of signal proteins from the mitochondria, activation of a class of specific intracellular proteinases, the caspases, and fragmentation of DNA. A consequence of apoptosis is alterations in the structure of the plasma membrane, including exposure of phosphatidylserine head groups on the outer leaflet of the lipid bilayer and appearance of new antigens. These changes serve as signals for phagocytosis of apoptotic bodies by macrophages or other cells.

Apoptosis is crucial to development of neoplasms. During tumour development cells die as a consequence of a failing energy supply as a result of competition with other mutant cells that are better adapted to the environment of the tumour. Apoptotic cells are also found in cardiac infarctions and are predominant in atherosclerotic lesions. Apoptosis may influence the development of the lesion, in particular its progress towards a stable or unstable condition. Unstable atheroclerotic plaques are associated with an increased risk of fragmentation of the plaque, in turn implying thrombi in other parts of the body.

Apoptosis and/or necrosis are also involved in the damage due to cerebral ischemia and degenerative diseases of the central nervous system, such as Alzheimer's and multiple sclerosis. In addition the processes are important in inflammations.

During the apoptotic process, the enzyme transglutaminase (protein-glutamine-(-glutamyl transferase) is activated. This enzyme catalyzes the exchange of the —NH₂ group of glutamine with the 6-amino group of lysine, releasing ammonia and forming protein cross-links, ultimate forming a network of densely cross-linked proteins. The functions of this enzyme in apoptosis are not entirely clear, but it may serve to ensure the coherence of cell contents, preventing their release during the late stages of apoptosis. In necrosis, intracellular transglutaminase is activated following influx of calcium ions.

Transglutaminases comprise a class of enzymes. The most familiar member is Factor XIIIa, which creates cross-links between fibrin molecules in blood clot formation. The other transglutaminases are usually lumped together as “tissue transglutaminases”. In common with Factor XIIIa, they require calcium concentrations in the millimolar range for activity. In normal cells, a “high intracellular calcium concentration” is of the order of 10⁻⁵ M, far below the minimum required to activate transglutaminases. Some tissue transglutaminases are active in dead cells that are permeable to calcium, for instance the keratinocytes. Others are secreted to function in cell adhesion or modification of the substrate.

Transglutaminases must be regarded as acting on two substrates, a lysine side chain and a glutamine side chain. Analogues of the lysine side chain can be very simple, for instance the straight-chain diamines putresceine and cadaverine, plus a wide range of monosubstituted derivatives, notably dansylcadaverine. In most assays, the glutamine side chain is part of a protein; the dipeptide benzyloxycarbonyl-L-glutamylglycine will also work as an acceptor. There are significant differences in specificity between various transglutaminases. A decapeptide amide, Leu-Gly-Leu-Gly-GlN-Gly-Lys-Val-Leu-GlyNH₂, has been found to be a good substrate for Factor XIIIa as well as for tissue transglutaminase from pig liver, but the activity for Factor XIIIa was lost on reversing the Val-Leu sequence.

It has now surprisingly been found that substrates of the enzyme transglutaminase are useful in contrast agents substrates in imaging of apoptosis and necrosis. The enzyme activity results in contrast agent products that are localised to the cells or tissues in question and/or have different contrast efficiency than the contrast agent substrate. Relevant diseases include neoplastic disease, including malignant as well as non-malignant tumours, cardiovascular diseases, including infarctions and thrombosis, and degenerative diseases of the central nervous system, such as Alzheimer's disease.

In the body, transglutaminase joins two different side chains of proteins, lysine and glutamine, forming an isopeptide bond. Accordingly, the substrates useful in imaging of apoptosis and necrosis fall into two distinct classes that may be roughly designated as “lysine mimics” and “glutamine-containing peptides”. The “lysine mimics” may be simply a primary amino group at the end of a straight hydrocarbon chain of four or more, preferably five or more, carbon atoms, with a reporter group at the other end. Larger substrates, including multivalent substrates with two or more alkylamino groups, are also provided. There may be advantages in including the alkylamino group as lysine, preferably as part of a peptide, in that different peptides may exhibit different activities towards various transglutaminases. For instance, a low activity towards Factor XIIIa may be desirable. In this document, “lysine” will be taken to mean the amino acid lysine as well as related amino acids possessing a straight chain of four or more carbons such as 2,4-diaminobutyric acid, ornithine, hydroxylysine, N(6)-methyl-lysine, N(2)-methyl-lysine, 2,7-diaminoheptanoic acid and so forth. D- as well as L-enantiomers are included.

Glutamine-containing peptides will always include the amino acid glutamine and/or homologues of glutamine possessing four or more carbon atoms, for instance asparagine, 2-amino-adipic acid-6-amide, glutamic acid analogues substituted with alkyl (e.g., methyl) on one or more nitrogens, e.g. glutamic acid 5-methylamide. It is generally recognised that the peptide must be at least a dipeptide, for instance GlN-Gly which is blocked at the N-terminal. The blocking group could be a reporter group, such as a chelate for nuclear imaging or MRI contrast, or a ¹⁹F-containing group for MRI contrast, or, for PET scans, a moiety that could easily be modified to comprise an ¹⁸F-atom. The selectivity of the substrate may be improved by using a longer peptide.

Caspases are intracellular proteases that become activated during the apoptotic process. They are a family of aspartate-directed proteases. Activation of caspases proceeds by a cascade mechanism. One of the last to be activated is caspase-3, and this proteinase should, accordingly, be a reliable indicator of commitment to the apoptotic process. The caspases cleave a number of important intracellular proteins, including several protein kinases, components of the DNA repair machinery, and structural elements of the cytoplasm and nucleus.

Most, if not all caspases, cleave at the carboxy terminus of an aspartate residue. This residue is preceded by a sequence of four to five amino acids that determine the specificity of each caspase (Talanian R V et al. (1997), J. Biol Chem. 272, 9677-9682). Many of these sequences contain another aspartate or glutamate residue, making them acidic. Valine or another amino acid containing an aliphatic hydrophobic side chain are also commonly encountered. Substrates for the fluorometric or colorimeric detection of caspase-3 in cultured cells have been made (Gurtu V. et al. (1997) Anal. Biochem. 251, 98-102). Substrates for caspases may be used according to the invention for identification of apoptosis.

A contrast agent substrate may comprise a contrast active element linked to an enzyme substrate, optionally by a linker/spacer. For such contrast agent substrates the chemical modification to a contrast agent product can involve a hydrolysis. In the following example reactions hydrolysis of different enzyme substrates for corresponding hydrolytic enzymes are shown. In these reactions the alkyl group could be alifatic, alicyclic, aromatic, substituted or non-substituted, linear or branched and may comprise from 1 to 50 atoms. The alkyl-group can be linked to a contrast active element, not shown in the reactions.

Phosphatases reaction (alkaline or acid):

Alkyl-O—PO₃²⁻→alkyl-OH+PO₄²⁻.

Aminopeptidase A reaction:

Alkyl-(NH)(Glu)ₙ→alkyl-NH₃₊+nGlu.

Aminopeptidase reaction:

4-alkyl-(C₆R¹R²R³R⁴)NH-amino acid-NH₂→4-alkyl-(C₆R₁R²R³R⁴)NH₂+amino acid,

in which R¹-R⁴ are electronegative groups (F, Cl, NO₂ etc.) in sufficient numbers to ensure that the amino group is minimally protonated at physiological pH. The change in charge will depend on the charge of the amino acid residue (e.g., Lys or Arg would result in the loss of two positive charges).

Carboxypeptidase reaction:

4-alkyl-(C₆H₄)-CO-amino acid-CO₂H→4-alkyl-(C₆H₄)-CO₂⁻+amino acid

Monoamine oxidase reaction:

4-alkyl-C₆H₄-CH₂-NH₃⁺+H₂O+O₂→4-alkyl-C₆H₄-CHO+H₂O₂+NH₄⁺

β-Glucuronidasereaction:

1-alkyl-β-O-glucuronic acid→alkyl-OH+glucuronic acid.

Analogous reactions may be devised for other enzymes such as e.g. galacturonidase or iduronidase.

The above concepts may for instance be applied in ultrasound imaging using contrast agents comprising microbubbles having a wall material that at least partly comprises molecules that are substrates for disease related enzymes. For this purpose the chemical chemical modification from a contrast agent substrate to a contrast agent product comprise a hydrolysis of peptides in the wall material. The alkyl group would then preferably be an aliphatic moiety comprising about 12-24 carbon atoms, such as myristyl, cetyl or stearyl. In certain instances, the alkyl group may be found as a substituent on a phenyl group, which may itself be substituted (e.g., 4-alkylphenyl-). This modification may result in better substrates for certain hydrolytic enzymes.

Besides hydrolytic cleavage there is a long list of chemical modifications that may occur when a contrast agent substrate is turned into a contrast agent product upon a enzymatic transformation. The following chemical modifications are included:

Hydrolytic cleavage:

Proteolysis

Extracellular: metalloproteinases, prostate-specific antigen, collagenases

Intracellular: lysosomal enzymes, proteasomes, calpain, caspases

Peptidases (carboxypeptidases, aminopeptidases)

Hydrolysis of phosphate esters (phospholipases C and D, phosphatases)

Hydrolysis of esters (lipases, esterases, phospholipases A and B, cholinesterases)

Amylases: Hydrolysis of glycogen

Glycosidases: glucuronidases, glucosidases, galactosidases, galacturonidases, mannosidases, sialidases, lactase

Hydrolysis of sulfate esters: arylsulfatase

Hydrolysis of nucleic acids: RNAses, DNAses

Chemical reactions of intermediate metabolism

The reactions catalyzed by Lactate dehydrogenase, glycogen phosphorylase, methylmalonyl-CoA mutase, lecithin:cholesterol acyltransferase, porphobilinogen deaminase and others

Biosynthetic

Formation of prostaglandins and thromboxanes from arachinonic acid

Synthesis of telomers (chromosome ends)

Farnesylation, geranylgeranylation, myristoylation, palmitoylation, GPI-anchoring and other hydrophobic modifications of proteins

DNA repair enzymes

Ubiquitination

Glycosylation of proteins, usually at asparagine or serine/threonine

Transfer of sugar moieties, usually from phosphate ester derivatives:

glucosyltransferases, fucosyltransferases, galactosyltransferases

Formation of thioether bonds: Gluthathione S-transferases

Formation of sulfate esters and sulfonamides: sulfo-transferases

Reactions involved in signalling pathways:

Nitric oxide synthetase

Formation of phosphate esters at serine, threonine or tyrosine in proteins:

protein kinases

Hydrolysis of phosphate esters in protein: protein phosphatases

Angiotensin converting enzyme

Endothelin converting enzyme

Deamination of neurotransmitters: monoamine oxidase

Cyclization of ATP: adenylate cyclase

Miscellaneous

Topoisomerases (DNA unwinding enzymes)

Hydroxylations of steroids and aromatic compounds, including detoxification reactions: CYP 17, cytochrome P-450

Several enzymes are associated with many different diseases. Below is a description of some of the enzyme targets, their ligands and some examples of contrast media substrates:

Cyclooxygenase (COX) is the key enzyme in the metabolism of arachidonic acid and formation of prostaglandins. There are at least two distinct isoforms of COX; COX 1 and COX 2. Non-steroid anti-inflammatory drugs (NSAIDs) inhibit COX. NSAIDs are among the widely prescribed drugs in the world. Recently selective COX-2 inhibitors have been marketed. These new agents show less unwanted side effects, for example gastric bleeding, compared with older NSAIDs like indometacin. COX including COX-2 play an important role in inflammation. Based on COX (COX-2) expression in CNS diseases like Alzheimer's disease and in some cancers, inhibitors of COX-2 might be useful in prevention or treatment of these conditions. The enzyme activity of COX-2 is, in other words, dependent on the tissue state and thereby an interesting enzyme to map to diagnose disease. FIG. 2 shows some examples on contrast agent substrates for mapping of COX activity. These contrast agents substrates will be substrates for the COX family of enzymes forming cyclopentanoid intermediates followed by prostaglandines and thromboxanes. The contrast agents are also substrates for oxidative enzymes forming leukotrienes and related compounds.

[image]

Telomerase is an important enzyme required for maintenance of chromosome ends during cell division. Telomerase is a ribonucleoprotein which catalyzes the formation of telo repeats represented by TTAGGG at the end of chromosomes in vertebrates. The activity of telomerase is increased in a large number of neoplastic diseases. Based on this elevated activity of telomerase in tumors this enzyme has gained interest as a potential cancer marker and as target for future anticancer therapy. Contrast agents for diagnosis of cancer based on telomerase activity can be contrast labeled nucleic acids.

Beside farnesyltransferase and geranylgeranyltransferase, several other enzymes mediate transfer of hydrophobic residues to proteins, or removal of such residues as myristic or palmitic acids. Examples are palmitoyl-protein transferase, myristoyl-protein transferase, glycosyl-phosophatidylinositol transferase, and palmitoyl-protein thioesterase. Their activities may be modified in specific diseases. Myristoyl-protein transferase activity is increased in colon cancer, glycosyl-phosophatidylinositol transferase is increased in certain protozoic infections and may be involved in prion diseases of the central nervous system. Table 1 lists enzymes that mediate hydrophobic modifications of proteins, the related diseases/conditions and processes involved.

TABLE 1
Diseases or conditions related to hydrophobic modifications of proteins

| Process | Enzyme/pathway | Disease/condition |
|---|---|---|
| Palmitoylation | Palmitoyl-protein | Infantile neuronal |
| | thioesterase | ceroid lipofuscinosis |
| proteolysis | Ubiquitin-proteasome | Metabolic acidosis |
| proteolysis | Ubiquitin-proteasome | neurodegerative |
| proteolysis | Ubiquitin-proteasome | Cancer |
| farnesylation | Farnesyl protein | medullablastoma |
| | transferase | |
| farnesylation | Farnesyl protein | Hepatic carcinogenesis |
| | transferase | |
| farnesylation | Farnesyl protein | Ceroid lipofuscinosis |
| | transferase | (Batten's disease) |
| Geranylgeranylation | Geranylgeranol | Prostatic hyperplasia |
| | transferase | |
| Myristoylation | Myristoyl protein | HIV infection |
| | transferase | |
| Myristoylation | N-myristoyl | Colon cancer |
| | transferase | |
| GPI-anchoring | GPI transferase | Paroxysmal nocturnal |
| | | hemoglobinuria |
| GPI-anchoring | GPI transferase | Carbonic anhydrase |
| | | deficiencies |
| | | (osteopetrosis) |
| GPI-anchoring | GPI transferase | Prion diseases of the |
| | | CNS |
| GPI-anchoring | GPI transferase | Protozoiasis |

Note: GPI, glycosyl-phosphatidylinositol

Ras proteins are guanine nucleotide-binding proteins that play an important role in the control of normal cell growth. An activation of these Ras proteins might result in uncontrolled cell growth and cancer. Ras proteins play an important role in development of approximately 30% of human cancers; including cancers in pancreas and colon. Ras proteins undergo several modifications to activate the protein. An activation of Ras proteins starts with attachment of the proteins to the inner surface of the plasma membrane. To be able to attach to the membrane the Ras proteins have to become more lipophilic. This first modification is a modification where an isoprenoid moiety, a C-15 group (farnesyl diphosphate, FDP) is covalently linked to Ras. This process is catalyzed by farnesyltransferase (FTase). This enzyme has during the last years been a popular target for potential anticancer drugs. Several FTase inhibitors have been identified. Contrast agents for detection of high activity of Ftase might be used to diagnose/adentify cancer at a very early stage. Typical contrast agents for detection of a high Ftase activity and for diagnosis of cancer related to activity of FTase can be such as contrast labeled isoprene derivatives, e.g. farnesyl diphosphate analogs or other substrate analogs as shown in FIG. 3. Contrast agents for FTase activity could be ¹¹-labelled or ¹⁸F-labelled FDP for PET, ^{99m}Tc-labelled for scintigraphy, F-labelled for MRI or gadolinium labelled/superparamagnetically labelled for MRI.

A closely related enzyme is geranylgeranyltransferase. As these enzymes recognize specific amino acid sequences, farnesylation or geranylgeranylation may be used to trap peptides or proteins inside cells.

[image]

Many phospholipases are important in signal transduction. Phospholipase A₂ liberates arachidonic acid from phopholipids, providing the substrate for synthesis of prostaglandins and thromboxanes, which are mediators in inflammation. An interesting form of phospholipase A₂, lipoprotein-associated phospholipase A₂, degrades the powerful mediator platelet activating factor and is expressed in large amounts in atherosclerotic lesions. This Phospolipase has been described by Hakkinen et al in Arteriosclerosis Thrombosis and Vascular Biology 19 (12): 2909-2917. Another phospholipase, phospholipase C_{β}, forms the two important intracellular messengers, diacylglycerol and inositol 1,4,5-trisphosphate, from phosphatidylinositol bisphosphate.

The genome cannot remain functional without a full complement of DNA repair enzymes (e.g., about ten thousand N-glycosidic bonds between base and deoxyribose are broken each day, either spontaneously or through damage). These are enzymes that comprise a heterogeneous assortment of activities: removal of altered bases, excision of damaged nucleotides, filling in of gaps in the nucleotide sequence and nucleotide mismatch repair. The last-mentioned activity is deficient in hereditary nonpolyposis colon cancer; nucleotide excision repair is deficient in xeroderma pigmentosum, causing a 2000-fold increase in the frequency of skin cancer on exposure to ultraviolet light.

Mutations in the DNA repair enzymes are extremely important in the development of cancer. Synthetic substrates, preferably altered poly-or oligonucleotides, may be devised. For instance, the 3-methyladenine DNA glycosylase would release an adenine labeled in the 3-position from a poly-or oligonucleotide. The altered base would presumably leave the cell or be degraded thus causing altered pharmacokinetic properties. Analogous substrates for excision enzymes might be devised. Delivery systems for poly-or oligonucleotides, e.g. cationic liposomes, are familiar to anyone skilled in the art.

Imaging of alterations in the activity of DNA repair enzymes will be valuable in the diagnosis of cancer. It will also be an important guide to treatment, as some cytotoxic drugs are toxic primarily to cells in which specific DNA repair enzymes are intact (e.g., triazenes are toxic to cells that possess nucleotide mismatch repair systems, including normal cells). Conversely, drugs that act by altering bases in DNA, such as alkylating agents, might be expected to be effective against cells that lack enzymes for removing altered bases. As tumour cell populations are unstable, their properties with respect to DNA repair enzymes cannot usually be predicted. Cassiman et al describes DNA repair systems in Introduction to Tumor Biology (I. De Wever, ed.), Leuven University Press, Leuven 1999.

Topoisomerases are nuclear enzymes which catalyze breaking of transient DNA strands allowing the cell to manipulate the topology of DNA. Topoisomerase enzymes are essential for DNA replication, transcription and other critical nuclear process in cells. There are two forms of the enzyme, topoisomerase I and topoisomerase II. These enzymes are present in all cells. Both topoisomerase I and topoisomerase II have been targets for antineoplastic drugs and several commercially available anticancer drugs. Contrast agents for diagnosis of cancer based on mapping of topoisomerase activity is e.g. contrast labeled nucleic acids, nucleic acid fragments or analogs thereof.

Turnover of intracellular proteins occurs mainly in two distinct classes of organelles, lysosomes and proteasomes. Entire sections of cytoplasm enter in lysosomes by the process of autophagy, and the components are broken down by the action of lysosomal enzymes such as the cathepsins; lipids and oligosaccharides are degraded by lipases and glycosidases, respectively. For many of these enzymes, synthetic substrates are well known and may be modified for use in imaging. The activity of the autophagic-lysosomal pathway is increased in neoplastic cells, including many tumors.

Proteolysis in the proteasome (a large multiprotein complex) is much more selective. Frequently, the process is initiated by conjugation of the protein to be degraded with another protein, ubiquitin, by ubiquitin conjugating enzymes that form an isopeptide bond. A peptide comprising the recognition sequence for ubiquitination plus a paramagnetic chelate (MRI contrast agent) might be anchored to ubiquitin, thus increasing its relaxivity by reducing its tumbling rate. —Conversely, relaxivity might be decreased by degradation of a chelate-labeled protein-ubiquitin complex, possibly including the removal of ubiquitin by one of the de-ubiquitinating enzymes.

The activity of the ubiquitin-proteasome pathway is increased in neurodegenerative diseases, cancer, and metabolic acidosis. The latter condition might also include the low-pH conditions that prevail in many solid tumors.

Matrix metalloproteinases, MMPs, are important enzymes playing a central role in different pathological conditions including cancer. Breakdown of extracellular matrix proteins are critical for local tumor growth and matrix metalloproteinases catalyzes this process. MMPs are a family of 17 zinc-dependent endopeptidases and these endopeptidases degrade essentially all extracellular matrix components. Tumor invasion including metastasis are often associated with increased expression of MMPs. This family of enzymes has therefore been popular targets for new potential anticancer drugs and several MMP inhibitors have been identified. Contrast agents for mapping of MMP activity can be any contrast labelled substrate for MMP. The structures of the MMPs vary, cleaving a great variety of substrates. Typical substrates that might be labeled with contrast agents are listed in List 6. Contrast agents can be covalently linked to these macromolecules using well-described technology.

### LIST 6

### Substrates For Human Matrix Metalloproteinase

Collagens, Proteoglycans, Laminin, Fibronectin, Gelatins, Elastin, Perlacan, Entactin, Vitronectin, Tenascin, Nidogen, Dermatan sulphate, pro TNF-∀, Vitronectin, Aggrecan, Transin, Decorin, Glycoproteins

MMP could also be used according to the invention as possible targets for vulnerable atherosclerotic plaques. Reliable methods for targeting vulnerable atherosclerotic plaques are currently missing. Vulnerable plaques tend to rupture and induce thrombosis, which may lead to occlusion of the vessel and acute myocardial infarction. As a further aspect of the invention it is suggested to detect MMP activity as targets for distinguishing between stable and unstable/vulnerable atherosclerotic plaques.

Degradation of the fibrous cap in the atherosclerotic plaque by MMPs destabilises the plaque and increases its vulnerability. The activity of these MMPs, or the new epitopes exposed after metalloproteinases digestion, could be targets for contrast agents.

Atherosclerotic lesions initially consist of subendohelial accumulation of macrophages, which subsequently develop into fibroproliferative lesions with accumulation of extracellular matrix. A fibrous cap rich in smooth muscle cells and extracellular matrix overlies a central core containing foamy macrophages, cholesterol crystals etc. Pathological studies have documented the presence of intense infiltration of macrophages at sites of plaque rupture. These macrophages synthesise and secrete a diverse array of proteolytic enzymes. The MMPs is one such family of proteolytic enzymes which is capable of degrading all macromolecular constituents of the extracellular matrix, which destabilizes the fibrous cap of the plaque and increases its vulnerability. Active synthesis and secretion of MMPs is identified in atherosclerotic coronary arteries from patients with unstable angina. Much lower levels were found in samples from patients with stable angina. It has been found that metalloproteinases represent targets that are able to distinguish between stable and unstable/vulnerable atherosclerotic plaques. Another embodiment of the invention is hence to use the activity of MMPs as a target for contrast agents according to the invention. One approach for measuring the activity of the MMPs involves a contrast agent coupled to a substrate for the metalloproteinases. The MMP contrast agent substrate is changed into a contrast agent product upon a chemical modification. The enzymatic activity alters the mobility of the contrast agent or preferably change thepharmacodynamic and/or pharmacokinetic properties. The metalloproteinases digest the extracellular matrix components at specific sites, exposing new epitopes, which could be possible targets for nuclear imaging.

Imaging of MMP activity in an atherosclerotic plaque could be done using e.g. magnetic resonance imaging or nuclear imaging, wherein a contrast agent is linked to a peptide cleavable by a MMP. After cleavage it is important that the contrast agent is being trapped in the atherosclerotic plaque. One solution to this is that the cleavage of the peptide leads to exposure of ligands for a receptor, e.g. a scavenger receptor, expressed on foamy macrophages. The contrast agent exposed to an active MMP would then be trapped in an atherosclerotic plaque due to endocytoses by the present macrophages. The described solution for trapping molecular contrast agents in the region of targeting could also be used for other enzyme activities related to other pathophysiological processes.

An atherosclerotic plaque prone to rupture is characterised by increased influx of macrophages producing MMPs capable of degrading the fibrous cap. Foamy macrophages are not specific for unstable atherosclerotic plaques and most of the receptors identified on foamy macrophages are also expressed by macrophages elsewhere in the body. MMP activity is found in physiological processes where tissue remodeling is happening, like tumour growth and atherosclerotic plaque disruption. Peng et al., 1999 Gene Therapy 6:1552-1557 used MMP activity for selective transduction of retroviral vectors into MMP-rich tumour xenografts in vivo. A chimeric envelope construct consisting of a MMP-cleavable linker fused to ligand for receptors on the tumour cells (CD40 or EGF-receptor) was incorporated in the viral coat. A similar approach could be used for magnetic resonance or nuclear imaging. Several cleavable peptides for MMPs have been described and peptide ligands for a macrophage exposed receptor, like scavenger receptors, can be found using phage display. An increased activity of acid phosphatase enzyme activity has been observed in different pathological conditions like prostate carcinoma, thrombocytoperia and some liver diseases. Contrast media for diagnosis of disease based on acid phosphatase are any contrast labelled substrate for this enzyme. FIG. 4 shows some examples on contrast media substrates for acid phosphatase.

[image]

Increased activity of alkaline phosphatase is observed in some diseases of the liver and bone diseases and in some other diseases like heart failure and bacterial infections. Substrates for alkaline phosphatase are similar to substrates for acid phosphatase. (See FIG. 4). Preferred modalities are MRI and nuclear medicine for these contrast agent substrates. The phosphate group in these contrast agent substrates will be transferred to a hydroxyl group.

Increased levels of ∀-amylase are associated with pancreatitis, intra-abdominal diseases and bacterial parotitis. Contrast agents for diagnosis of disease based on abnormal ∀-amylase activity can be such as paramagnetically labelled cross-linked starch microspheres described by P. Rongved et al in Carbohydrate Research 214 (1991) 325-330.

Increased levels of β-glucuronidase are associated with several diseases including diabetes mellitus, renal diseases, pancreatic cancer and liver diseases. Typical contrast agents for diagnosis of diseases based on abnormal activity of β-glucuronidase are conjugates between a contrast active moiety and glucuronic acid directly or through a spacer.

Lipase cleaves triglycerides into fatty acids and diglycerides. Increased levels of lipase are associated with acute pancreatitis and some other diseases located in the abdomen. Contrast agents for mapping of lipase activity can be contrast labelled triglycerides. Examples of substrates are contrast labeled triglycerides.

CYP 17 or 17 ∀-hydroxylase/17,20-lyase is an enzyme catalyzing the biosynthesis of androgens from pregnane precursors. Inhibition of this enzyme preventing formation of androgens may provide effective treatment of prostate cancer and is now an attractive research area for development of new anticancer agents. FIG. 5 lists some examples of contrast agentsubstrates for diagnosis of disease based on CYP 17 activity. Relevant modalities for these contrast agent substrates are MRI and nulear medicine, recpectively. A hydroxylation of the 17 position will happen in vivo during the enzymatic transformation.

[image]

Inherited defects in enzyme molecules are by far the largest category of heritable diseases. As expected, the kind and severity of disease varies greatly. In some populations, one individual in a hundred may be affected by a specific heritable enzyme deficiency. Many of the enzymes given in List 7 have been studied in detail, see Scriver et al in “The metabolic basis of inherited disease”, 6^{th} Edn., McGraw-Hill, New York 1989. Artificial substrates for these enzymes are available and these may be modified for imaging purposes. The patient may present with neurological symptoms, but the primary affected organ could be the liver. Thus, it is frequently important to localize the areas in which the enzyme was not expressed.

### LIST 7

### Enzymes That Are Known To Be Defective In Various Inherited Diseases.

1. Enzymes causing pharmacogenic disorders

Isoniazid acetylase

Pseudocholinesterase

Glucose 6-phosphate dehydrogenase

2. Disorders of carbohydrate metabolism

Fructokinase

Fructose 1,6-diphosphate aldolase B

Fructose 1,6-diphosphatase

Glucose 6-phosphatase

Glucose 6-phosphate translocase

α-Glucosidase (lysosomal)

Amylo-1,6- glucosidase

Amylo-1,4:1,6- glucantransferase

Gycogen phosphorylase

Phosphorylase b-kinase

Phosphofructokinase

Glycogen synthase

Phosphoglycerate kinase

Phosphoglycerate mutase

Lactate dehydrogenase

Glucose phosphate isomerase

Galactose-1-phosphate uridyltransferase

Galactokinase

Uridine diphosphate galactose 4-epimerase

L-xylulose reductase

3. Disorders of amino acid metabolism

Phenylalanine hydroxylase

Dihydropteridine reduktase

Guanosine triphosphate cyclohydrolase

6-Pyruvoyl tetrahydropterin synthase

Fumarylacetoacetate hydrolyase

Maleylacetoacetate isomerase

Tyrosine aminotransferase

Urocanase

Histidase

Proline oxidase

Δ-Pyrrolidine-5-carboxylate dehydrogenase

4-Hydroxy-L-proline-oxidase

Peptidase D

Omithine-δ-aminotransferase

Carbamyl phosphate synthase

Omithine transcarbamylase

Argininosuccinic acid synthase

Argininosuccinic acid synthase

Arginase

α-Aminoadipic semialdehyde synthase

Cysthathionine β-synthase

α-Cystathionase

Methionine adenosyltransferase

Sarcosine dehydrogenase

Dihydropyrimidine dehydrogenase

β-Alanine-pyruvate transaminase

R-β-Aminoisobutyrate-pyruvate transaminase

Glutamic acid decarboxylase

GABA-α-Ketoglutarate transaminase

Succinic semialdehyde dehydrogenase

Carnosinase

4. Disorders of metabolism of organic acids

Homogentisic acid oxidase

Isovaleryl-CoA dehydrogenase

3-Methylcrotononyl-CoA carboxylase

3-Methylglutaconyl-CoA hydratase

Mevalonate kinase

2-Methylacetoacetyl-CoA thiolase

3-Hydroxyisobutyryl-CoA deacylase

Propionyl-CoA carboxylase

Methylmalonyl-CoA mutase

ATP:Cobalamin adenosyltransferase

Glutaryl-CoA dehydrogenase

2-Ketoadipic acid dehydrogenase

Glutathione synthetase

5-Xxoprolinase

γ-Glutamylcysteine synthetase

δ-Glutamyl transpeptidase

Cytochrome oxidase

Fumarase

Pyruvate carboxylase

Long-chain acyl-CoA dehydrogenase

Medium-chain acyl-CoA dehydrogenase

Short-chain acyl-CoA dehydrogenase

Electron transfer flavoprotein:ubiquinone oxidoreductase

Alanine:glyoxylate aminotransferase

D-Glycerate dehydrogenase

Glycerol kinase

5. Disorders of metabolism of purines and pyrimidines

PP-Ribose-P synthetase

Hypoxanthine-guanine phosphoribosyltransferase

Adenine phosphoribosyltransferase

Adenosine deaminase

Purine nucleoside phosphorylase

Myoadenylate deaminase

Xanthine dehydrogenase

UMP synthase

Pyrimidine 5′nucleotidase

Dihydropyrimidine dehydrogenase

6. Disorders of lipid metabolism

Lipoprotein lipase

Lecithin:cholesterol acyltransferase

26-hydroxylase (cholesterol)

7. Disorders of metabolism of porphyrins and heme

δ-Aminolevulinic acid dehydratase

Porphobilinogen deaminase

Uroporphyrinogen cosynthase

Uroporphyrinogen decarboxylase

Coproporphyrinogen oxidase

Protoporphyrinogen oxidase

Ferrochelatase

Bilirubin UDPglucuronyl transferase

Phytanic acid α-hydroxylase

Catalase

8. Disorders of lysosomal enzymes

α-L-iduronidase

Iduronate sulfatase

Heparan-N-sulfatase

α-N-acetylglucosaminidase

Acetyl-CoA-α-glucosaminide acetyltransferase

Acetylglucosamine 6-sulfatase

Ggalactose 6-sulfatase

β-Galactosidase

N-Acetylgalactosamine 4-sulfatase

β-Glucuronidase

UDP:N- Acetylglucosamine:lysosomal enzyme N-acetylglucosaminyl-1-phosphotransferase

α-Mannosidase

α-Neuraminidase

Aspartylglucosaminidase

α-L-Fucosidase

Aacid lipase

Acid ceramidase

Sphingomyelinase

Glucocerebrosidase

Galactosylceramidase

Steroid sulfatase

Arylsulfatase

α-Galactosidase

α-N-Acetylgalactosaminidase

Acid β-galactosidase

β-Hexosaminidase

9. Disorders of metabolism of hormones

Ssteroid 21-hydroxylase

Steroid 5α-reductase

3-β-Hydroxysteroid sulfatase

25(OH)D₃-1-α-hydroxylase

10. Disorders of metabolism of vitamins

Methylene tetrahydrofolate reductase

Glutamate formiminotransferase

Holocarboxylase synthetase

Biotinidase

11. Disorders of blood

Cytochrome b₅ reductase

Pyruvate kinase

Hexokinase

Glucosephosphate isomerase

Aldolase

Triosephosphate isomerase

Phosphoglycerate kinase

2,3 -Diphosphoglyceromutase

6-Phosphogluconate dehydrogenase

Gluthathione peroxidase

Gluthathione reductase

Gluthathione synthetase

γ-Glutamylcysteine synthetase

12. Disorders of the immune system

Adenosine deaminase

Pyrimidine nucelotidase

Myeloperoxidase

NADPH oxidase

13. Disorders of connective tissues

Lysyl hydroxylase

Collagenase

Alkaline phosphatase

Carbonic anhydrase

14. Disorders of skin

Tyrosinase

15. Disorders of digestion

Lactase

Trehalase

Preferred enzymes that are known to be defective in various inherited diseases are glucose 6-phosphate dehydrogenase, lactate dehydrogenase, L-xylulose reductase, phenylalanine hydroxylase, fumarylacetoacetate hydrolyase, histidase, peptidase D (prolidase), carbamyl phosphate synthase, ornithine transcarbamylase, argininosuccinic acid synthase, argininosuccinase, arginase, carbamyl phosphate synthase, ornithine transcarbamylase, argininosuccinic acid synthase, arginase, methylmalonyl-CoA mutase, ATP:cobalamin adenosyltransferase, 2-ketoadipic acid dehydrogenase, medium-chain acyl-CoA dehydrogenase, hypoxanthine-guanine phosphoribosyltransferase, myoadenylate deaminase, xanthine dehydrogenase, porphobilinogen deaminase, catalase, α-L-iduronidase, iduronate sulfatase, heparan-N-sulfatase, α-N-acetylglucosaminidase, acetyl-CoA-α-glucosaminide acetyltransferase, acetylglucosamine 6-sulfatase, glucocerebrosidase, arylsulfatase, α-galactosidase, acid B-galactosidase, B-hexosaminidase, steroid 21-hydroxylase, 3-β-hydroxysteroid sulfatase, biotinidase, pyruvate kinase, and myeloperoxidase. Most preferred are glucose 6-phosphate dehydrogenase, phenylalanine hydroxylase, argininosuccinase, medium-chain acyl-CoA dehydrogenase, hypoxanthine-guanine phosphoribosyltransferase, lipoprotein lipase, steroid 21-hydroxylase, and myeloperoxidase.

The contrast agent substrates according to the present invention can be water-soluble or water-insoluble molecules, e.g. compounds with limited solubility in water so that the compounds have to be administered as a powder or a suspension. The molecular weight of the contrast agents varies with disease and enzyme(s) associated with the disease. The molecular weight of the contrast agents can be low (50-2000) or high (above 2000).

The contrast agent substrates according to the present invention are synthetic organic compounds, naturally occurring compounds or semi-synthetic compounds labeled with at least one contrast active element. In the most preferred compounds, the contrast active element does not participate in the enzymatic transformation. The contrast agents are prepared synthetically/semi-synthetically using well-known synthetic transformation or by conjugation of the contrast active element to an enzyme substrate using well known methods. In the last case, the contrast active part can be directly conjugated to for instance known enzyme substrates or can be conjugated to substrates through spacer arms, e.g. diaminoalkyl spacers and PEG-spacers. Techniques for conjugation are well known in the literature, for instance in publications in J. Bioconjugate Chemistry.

Contrast agents according to the present invention can for instance have elements from peptides, peptido-mimetics, fatty acids, proteins, carbohydrates or biological precursors thereof. Contrast agents according to the present invention usually contain one or more of the following functional groups; alcohols, phenols, esters including esters with other acids than carboxyxclic acids, amides, amines, mercapto-groups, aromatic rings and heterocyclic ring systems. The overall structure of the contrast agents can be cyclic or linearWhere the contrast agent or a component thereof carries an overall charge, it may be used in the form of a salt with a physiologically acceptable counterion, for example an ammonium, substituted ammonium, alkali metal or alkaline earth metal cation or an anion deriving from an inorganic or organic acid.

The diagnostic agents of the present invention may be formulated in conventional pharmaceutical or veterinary parenteral administration forms, e.g. suspensions, dispersions, etc., for example in an aqueous vehicle such as water for injections.

Such compositions may further contain pharmaceutically acceptable diluents and excipients and formulation aids, for example stabilizers, antioxidants, osmolality adjusting agents, buffers, pH adjusting agents, etc.

The most preferred formulation is a sterile solution of suspension for intravascular administration or for direct injection into area of interest.

Where the agent is formulated in a ready-to-use form for parenteral administration, the carrier medium is preferably isotonic or somewhat hypertonic.

Where the particulate agent comprises a chelate or salt of an otherwise toxic metal species, e.g. a heavy metal ion, it may be desirable to include within the formulation a slight excess of a chelating agent, e.g. as discussed by Schering in DE-A-3640708, or more preferably a slight excess of the calcium salt of such a chelating agent.

The dosage of the diagnostic agents of the invention will depend upon the imaging modality, the contrast generating species and the means by which contrast enhancement occurs.

In general however dosages will be between 1/10 and 10 times the dosage conventionally used for the selected contrast generating species or analogous species in the same imaging modality. Even lower doses may also be used.

While the present invention is particularly suitable for methods involving parenteral administration of the particulate material, e.g. into the vasculature or directly into an organ or muscle tissue, intravenous administration being especially preferred, it is also applicable where administration is not via a parenteral route, e.g. where administration is transdermal, nasal, sub-lingual or is into an externally voding body cavity, e.g. the gi tract, the bladder, the uterus or the vagina. The present invention is deemed to extend to cover such administration.

The disclosures of all the documents mentioned herein are incorporated by reference. The following examples are illustrative only and not intended to be limiting. Other features and advantages of the invention will be apparent from the detailed description and from the claims.

### EXAMPLE 1

### A Matrix Metalloproteinase (MMP) Substrate For MRI. Mapping Of MMP-7 Activity

a) Synthesis of 1,4,7-tris(carboxymethyl-tert-butyl ester)-1,4,7,10-tetraazacyclododecane.

The synthesis of 1,4,7-tris(carboxymethyl-tert-butyl ester)-1,4,7,10-tetraazacyclododecane from now on called DO3A-TBE was carried out according to the procedure of L. Schulze & A. R. Buls (example 13 in WO 96/28433; PCT/GB96/00464). Based on H-NMR & C-NMR analysis DO3A-TBE was isolated as its mono HBr salt, protonated at N10.

b) Synthesis of (4,7,10-Tris-carboxymethyl-1,4,7,10-tetraaza-cyclododec-1-yl)-acetyl-Cys-Gly-Pro-Leu-Gly-Leu-Leu-Ala-Arg-OH

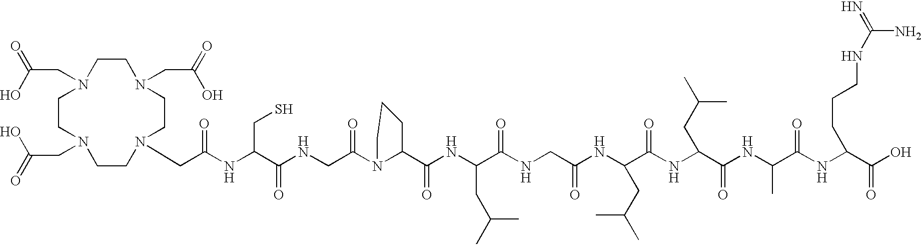

The peptide component was synthesised on a ABI 433A automatic peptide synthesiser starting with Fmoc-Arg(Pmc)-Wang Resin on a 0.1 mmol scale using 1 mmol amino acid cartridges. The amino acids were pre-activated using HBTU before coupling. An aliquot of the peptide resin was then transferred to a clean round bottom flask and 1 mmol of N-methyl morpholine in DMF (5 ml) added followed by 1 mmol of chloroacetyl chloride. The mixture was gently shaken until Kaiser test negative. Following extensive washing with DMF the resin was once again suspended in DMF (5 mL) and 1 mmol DO3A-TBE predissolved in 5 mL DMF containing 1 mmol Triethylamine added. The mixture was heated to 50 C. for 16 hours then excess reagents filtered off. Following extensive washing with DMF, DCM and diethyl ether then air drying, the peptide and side-chain protecting groups were simultaneous removed in TFA containing TIS (5%), H₂O (5%), and phenol (2.5%) for two hours. Excess TFA was removed in vacuo and the peptide precipitated by the addition of diethyl ether. 40 mg of crude peptide was obtained following trituration with diethyl ether and air drying. The crude peptide was purified by preparative HPLC (Luna C18 250×21.2 mm column) using a gradient of 10-50% B, where A=H₂O/0.1% TFA and B=CH₃CN/0.1% TFA, over 40 min at a flow rate of 9 ml/min. After lyophilisation 12 mg of pure material was obtained (Analytical HPLC: Gradient, 5-50% B where A=H₂O/0.1 % TFA and B=CH₃CN/0.1% TFA; column, Luna C 18 50×4.6 mm; detection, UV 214 nm; product retention time, 5.8 min). Further product characterisation was carried out using ESMS spectrometry: expected, M+H at 1285, found, at 1285).

A Gd-chelate of the above product could easily been made by conventional methods. Such product could be used as a contrast agent substrate for matrix metalloproteinase 7 (MMP-7) in MRI.

### Reaction Of 1,4,7-tris(carboxymethyl-tert-butyl ester)-1,4,7,10-tetraazacyclododecane with MMP-7

Recombinant matrix metalloproteinase-7 (MMP-7) was purchased from R&D Labs, (Abingdon, UK). Ten micrograms of the enzyme was dissolved in 100 μl 50 mM tris buffer, pH 7.4, containing containing 10 mM CaCl₂ and 150 mM NaCl. The enzyme was activated by the addition of 2 μl of 50 mM aminophenylmercuric acetate and incubation at 37° C. for 1 hour. 0.97 mg of 1,4,7-tris(carboxymethyl-tert-butyl ester)-1,4,7,10-tetraazacyclododecane was dissolved in 100 μl of the same buffer. One-half of this solution was added to the activated MMP-7, the other half was added to 0.1 ml of buffer (control incubation). The samples were incubated at 37° C. for 1 hour. The presence of the expected reaction product, the peptide Leu-Ala-Arg in the reaction mixture was determined by LC-MS using a C18 column eluted with acetonitrile/water/ 0,1% formic acid and full scan detection in a narrow mass range.

### EXAMPLE 2

### Synthesis Of Pn216-Succininyl-Gly-His-His-Pro-His-Gly-Pro-Ile-Cys(Et)-Phe-Phe-Arg-Leu-OH. A Cathepsin D Substrate For Nuclear Medicine Imaging.

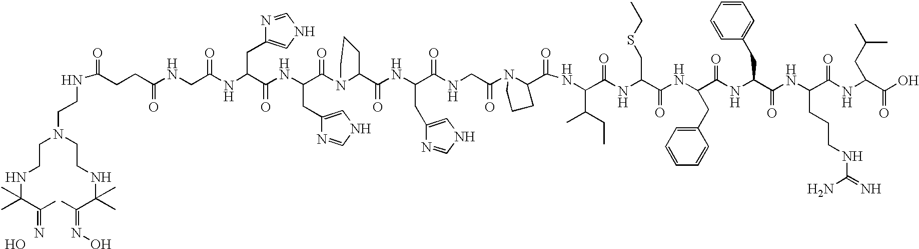

where Pn216 =Bis[(1,1 -dimethyl-2-N-hydroxyimine propyl)aminoethyl]-2-aminoethyl amine

The peptide component was synthesised on an ABI 433A automatic peptide synthesiser starting with Fmoc-Leu-Wang Resin on a 0.1 mmol scale using 1 mmol amino acid cartridges. The amino acids were pre-activated using HBTU before coupling and the resin capped using succinic acid anhydride yielding a resin bound acid function. On-resin activation using 3 equivalents of PyAOP, HOAt and N-methylmorpholine was carried out in DMF (10 mL) for 10 minutes before the addition of a solution in DMF (5 mL) of Pn216—Bis[(1,1-dimethyl-2-N-hydroxyimine propyl)aminoethyl]-2-aminoethyl amine. The coupling reaction was allowed to proceed for 4 hours then the resin washed with DMF, DCM and diethyl ether before air-drying. The peptide and side-chain protecting groups were simultaneous removed in TFA containing TIS (5%), H₂O (5%), and phenol (2.5%) for two hours. Excess TFA was removed in vacuo and the peptide precipitated by the addition of diethyl ether.

The crude peptide was purified by preparative HPLC (Luna C18 250×10 mm column) using a gradient of 5-50% B, where A=H₂O/0.1 % TFA and B=CH₃CN/0.1% TFA, over 30 min at a flow rate of 5 ml/min. After lyophilisation 3 mg of pure material was obtained (Analytical HPLC: Gradient, 5-50% B where A=H₂O/0.1% TFA and B=CH₃CN/0.1% TFA; column, Luna C 18 50×4.6 mm; detection, UV 214 nm; product retention time, 8.6 min). Further product characterisation was carried out using ESMS spectrometry: expected, M+H at 1973, found, at 1973.

The Tc-chelate of the above compound could easily been made by conventional methods. The chelate could be used as a contrast agent substrate for Cathepsin D in nuclear medicine imaging.

### EXAMPLE 3

### Gadolinium 1,4,7-tris(carboxymethyl)-10-(benzyl)carbamoyl-1,4,7,10-tetraazacyclododecane (4a)

The title compound was prepared by the following steps:

1,4,7-Tris(tert-butylcarbonylmethyl)-10-(benzyl)carbamoyl-1,4,7,10-tetraazacyclododecane (2a)

Benzyl isocyanate (1.33 g, 10 mmol) in DMF (100 ml) was added to 1,4,7-tris(tert-butylcarbonylmethyl)-1,4,7,10-tetraazacyclododecane (5.15 g, 10 mmol) and triethylamine (5.06 g, 50 mmol) dissolved in DMF (30 ml). The reaction mixture was stirred at ambient temperature overnight and evaporated in vacuo. The residue was submitted to flash chromatography (hexane/ethyl acetate/triethylamine, 7:3:1) to give the product as oil. Yield: 4.32 g (67%).

¹H NMR (CDCl₃): 7.88 (broad s, 1H), 7.35-7.13 (m, 5H), 4.34 (s, 2H), 3.35-3.25 (m, 4H), 3.17 (s, 6H), 3.00-2.85 (m, 4H), 2.75-2.58 (m, 8H), 1.42 (s, 9H), 1.39 (s, 18H).

1,4,7-Tris(carboxymethyl)-10-(benzyl)carbamoyl-1,4,7,10-tetraazacyclododecane (3a)

1,4,7-Tris(tert-butylcarbonylmethyl)-10-(benzyl)carbamoyl-1,4,7,10-tetraazacyclododecane (1.29 g, 2 mmol) was added trifluoroacetic acid (10 ml) and stirred under an argon atmosphere at ambient temperature for 6 h. before the reaction mixture was evaporated in vacuo. The residue was added water (5 ml) and evaporated in vacuo. The residue was re-dissolved in water and evaporated to dryness two more times. The crude material was purified by column chromatography using poly(4-vinylpyridine) macroreticular (Reillex 425) resin. The column was eluted with water and the fractions containing the products was combined and concentrated in vacuo. White solid material was obtained by dissolving the residue in methanol (2 ml) followed by diethyl ether precipitation and drying in vacuo at 50° C. overnight. Yield: 0.89 g (93%).

¹H NMR (D₂O): 7.31-7.20 (s, 5H), 4.20 (s, 2H), 3.85 (broad s, 4H), 3.61 (broad s, 4H), 3.41 (broad s, 6H), 3.27 (broad s, 4H), 2.83 (broad s, 4H).

Gadolinium 1,4,7-tris(carboxymethyl)-10-(benzyl)carbamoyl-1,4,7,10-tetraazacyclododecane (4a)

A suspension of gadolinium(III)oxide (0.27 g, 0.75 mmol) and 1,4,7-tris(carboxymethyl)-10-(benzyl)carbamoyl-1,4,7,10-tetraazacyclododecane (0.97 g, 1.50 mmol) in water was heated to 90° C. for 5 h. The resulting solution was cooled to room temperature and added cation-exchange resin (Amberlite IR 120/H⁺-form) and anion-exhange resin (Amberlite IRA 67/OH⁻-form). After stirring for 30 min. The resin was collected by filtration (Millipore HAWP 0.45 μm) and the filtrate was evaporated in vacuo to give the product as white crystalline material. Yield: 0.55 g (58%).

### EXAMPLE 4

### Gadolinium 1,4,7-tris(carboxymethyl)-10-(p-tolyl)carbamoul-1,4,7,10-tetraazacyclododecane (4b)

The title compound was prepared by the following steps:

1,4,7-Tris(tert-butylcarbonylmethyl)-10-(p-tolyl)carbamoyl-1,4,7,10-tetraazacyclododecane (2b)

p-Tolyl isocyanate (0.67 g, 5 mmol) in DMF (50 ml) was added to 1,4,7-tris(tert-butylcarbonylmethyl)-1,4,7,10-tetraazacyclododecane (2.57 g, 5 mmol) and triethylamine (2.53 g, 25 mmol) dissolved in DMF (20 ml). The reaction mixture was stirred at ambient temperature overnight and evaporated in vacuo. The residue was submitted to flash chromatography (hexane/ethyl acetate/triethylamine, 7:3:1) to give the product as oil. Yield: 2.63 g (81%).

¹H NMR (MeOD): 9.72 (s, 1H), 7.30-7.04 (q, J=8.4 Hz, 4H), 3.55-3.42 (m, 4H), 3.39 (s, 6H), 3.26 (s, 2H), 3.20-3.08 (m, 4H), 2.93-2.75 (m, 8H), 2.30 (s, 3H), 1.48 (s, 9H), 1.46 (s, 18H).

1,4,7-Tris(carboxymethyl)-10-(p-tolyl)carbamoyl-1,4,7,10-tetraazacyclododecane (3b)

1,4,7-Tris(tert-butylcarbonylmethyl)-10-(p-tolyl)carbamoyl- 1,4,7,10-tetraazacyclododecane (1.02 g, 1.57 mmol) was added trifluoroacetic acid (5 ml) and stirred under an argon atmosphere at ambient temperature for 6 h. before the reaction mixture was evaporated in vacuo. The residue was added water (5 ml) and evaporated in vacuo. The residue was re-dissolved in water and evaporated to dryness two more times. The crude material was purified by column chromatography using poly(4-vinylpyridine) macroreticular (Reillex 425) resin. The column was eluted with water and the fractions containing the products was combined and concentrated in vacuo. White solid material was obtained by dissolving the residue in methanol (2 ml) followed by diethyl ether precipitation and drying in vacuo at 50° C. overnight. Yield: 0.70 g (94%).

¹H NMR (D₂O): 7.13 (s, 4H), 4.02 (broad s, 4H), 3.52-3.26 (broad m, 14H), 3.07 (broad s, 4 H), 2.22 (s, 3H).

Gadolinium 1,4,7-tris(carboxymethyl)-10-(p-tolyl)carbamoyl-1,4,7,10-tetraazacyclododecane (4b)

A suspension of gadolinium(III)oxide (0.27 g, 0.75 mmol) and 1,4,7-tris(carboxymethyl)-10-(p-tolyl)carbamoyl-1,4,7,10-tetraazacyclododecane (0.97 g, 1.50 mmol) in water was heated to 90° C. for 5 h. The resulting solution was cooled to room temperature and added cation-exchange resin (Amberlite IR 120/H⁺-form) and anion-exhange resin (Amberlite IRA 67/OH⁻-form). After stirring for 30 min. The resin was collected by filtration (Millipore HAWP 0.45 μm) and the filtrate was evaporated in vacuo to give the product as white crystalline material. Yield: 0.45 g (47%).

### EXAMPLE 5

### Metabolic Experiments/Relaxometry. Oxidative Transformation Of Gd-chelates

A saline-solution of the respective Gd-chelates, 4a and 4b described in example 3 and 4, (2 mM) were incubated at 37° C. with 1, 10 or 50% (v/w) of homogenized liver from cattle. The T₁-relaxation times were measured at 20 MHz (37° C.) after 0, 1, 24, 36 and 60 h. as given in table 2. The T₁-relaxation time increased exponentially for all the homogenates. No change in the T₁-relaxation time for liver homogenates (50, 10 or 1% (liver/saline w/v)) without Gd-chelates was observed.

TABLE 2
T₁-relaxation time in different liver homogenate containing
a Gd-chelate as a function of incubation time.

T₁-relaxation time (ms)

| | Gd-chelate (4a) | | | Gd-chelate (4b) | | |
|---|---|---|---|---|---|---|
| | 50% | 10% | 1% | 50% | 10% | 1% |
| time (h) | liver | liver | liver | liver | liver | liver |
| 0 | 129 | 125 | 136 | 120 | 103 | 128 |
| 1 | 132 | 132 | 137 | 125 | 108 | 130 |
| 24 | 158 | 175 | 142 | 193 | 165 | 134 |
| 36 | 165 | 182 | 144 | 200 | 157 | 135 |
| 60 | 175 | 184 | 145 | 215 | 179 | 137 |

The chelates change contrast efficacy (T₁-relaxation time) as a result of the oxidative transformation.

### EXAMPLE 6

### 1,4,7-Tris(carboxymethyl)-10-((4-methyl)benzamide Phenylalanine Triethylenglycolmonomethylether Ester)-1,4,7,10-tetraazacyclododecane. Mapping Of Esterase Activity.

The title compound was prepared by the following steps.

4-(Chloromethyl)benzamide phenylalanine tert-butyl ester

4-(Chloromethyl)benzoylchloride (1.89 g, 10 mmol) and triethylamine (2.02 g, 20 mmol) dissolved in dichloromethane (22 ml) was added phenylalanine tert-butyl ester hydrochloride (2.58 g, 10 mmol) over a period of 4 h at 0° C. The reaction mixture was stirred overnight and extracted with aqueous HCl (5%, 2×50 ml) and aqueous Na₂CO₃ (5%, 2×50 ml). The organic phase was dried (MgSO₄) and evaporated in vacuo. Flash chromatography (methanol/chloroform, 1:200) gave the product as a white solid material. Yield: 1.99 g (76%).

¹H NMR (CDCl₃): 7.77 (d, J=8.3 Hz, 2H), 7.48 (d, J=8.4 Hz, 2H), 7.34-7.20 (m, 5H), 5.00(q, J=7.4 Hz, 1H), 4.64(s, 2H), 3.27 (d, J=5.7 Hz, 2H), 1.49 (s, 9H).

4-(Chloromethyl) benzamide phenylalanine

4-(Chloromethyl)benzamide phenylalanine tert-butyl ester (0.87 g, 2.3 mmol) was stirred in trifluoroacetic acid (10 ml) for 30 min. at ambient temperature before the mixture was evaporated in vacuo. The residue was dissolved in water (20 ml) and evaporated to dryness in vacuo. The last process was repeated once giving a white solid material. Yield: 0.67 g (92%).

¹H NMR (CDCl₃): 7.71 (d, J=8.3 Hz, 2H), 7.46 (d, J=8.3 Hz, 2H), 7.37-7.21 (m, 5H), 6.78 (d, J=7.4 Hz, 1H), 5.12 (q, J=7.3 Hz, 1H), 4.62 (s, 2H), 3.35 (m, 2H).

4-(Chloromethyl) benzamide phenylalanine triethylenglycolmonomethylether ester

4-(Chloromethyl) benzamide phenylalanine (0.62 g, 1.95 mmol) was added triethylenglycolmonomethylether (7.5 ml) and aqueous HCl (37%, 0.1 ml). The mixture was heated to 60° C. and stirred overnight. The reaction mixture was added chloroform (10 ml) and water (10 ml) and the phases were separated. The organic phase was extracted with water (2×10 ml) and evaporated in vacuo. The residue was subjected to flash chromatography (methanol/chloroform, 5:300) to give the product as colourless oil that solidified upon drying. Yield: 0.89 g (99%).

¹H NMR (CDCl₃): 7.62 (d, J=8.3 Hz, 2H), 7.30 (d, J=8.3 Hz, 2H), 7.19-7.06 (m, 5H), 4.98 (q, J=5.6 Hz, 1H), 4.48 (s, 2H), 4.19 (m, 2H), 3.40 (m, 15H).

1,4,7-Tris(tert-butylcarbonylmethyl)-10-((4-methyl)benzamide phenylalanine triethylenglycolmonomethylether ester)-1,4,7,10-tetraazacyclododecane (y)

1,4,7-Tris(tert-butylcarbonylmethyl)-1,4,7,10-tetraazacyclododecane (0.85 g, 1.42 mmol), (4-chloromethyl) benzamide phenylalanine triethylenglycolmonomethylether ester (0.66 g, 1.42 mmol) and K₂CO₃ (1.96 g, 14.2 mmol) in DMF (10 ml) was heated to 60° C. and stirred for 2 h. The reaction mixture was filtered and evaporated in vacuo. Flash chromatography (methanol/chloroform/ammonia 25%, 2:8:0.1) gave the product as colourless material. Yield: 0.48 g (36%).

¹H NMR (MeOD): 7.95 (s, 1H), 7.80 (d, J=7.8 Hz, 2H), 7.62 (d, J=7.8 Hz, 2H), 7.31-7.22 (m, 5H), 4.89 (q, J=5.3 and 3.4Hz, 1H), 4.78 (s, 4H), 4.28 (t, J=1.4Hz, 2H), 3.70-2.75 (m, 37H), 1.52-1.47 (s, 27H).

1,4,7-Tris(carboxymethyl)-10-((4-methyl)benzamide phenylalanine triethylenglycolmonomethylether ester)-1,4,7,10-tetraazacyclododecane

1,4,7-Tris(tert-butylcarbonylmethyl)-10-((4-methyl)benzamide phenylalanine triethylenglycolmonomethylether ester)-1,4,7,10-tetraazacyclododecane (0.48 g, 0.5 mmol) was added trifluoroactetic acid (6.5 ml) and stirred at ambient temperature for 1 h. The reaction mixture was evaporated in vacuo. The residue was dissolved in water (5 ml) and evaporated to dryness in vacuo. The last process was repeated once. The crude product was dissolved in methanol (1.5 ml) and precipitated with diethyl ether to give the product as a white solid material. Yield: 200 mg.

¹H NMR (D₂O): 8.13 (t, J=8.2 Hz, 2H), 8.04-7.99 (m, 3H), 7.80-7.72 (m, 5H), 5.32 (q, J=6.3 Hz, 1H), 4.74-4.71 (m, 5H), 4.33-3.43 (m, 37H).

A Gd-chelate of the above compound could easily been made by conventional methods.

Reaction of the Esterase substrate 1,4,7-Tris(carboxymethyl)-10-((4-methyl)benzamide phenylalanine triethylenglycolmonomethylether ester)-1,4,7,10-tetraazacyclododecane with carboxylesterease

Ten mg of the esterase substrate was dissolved in 50 μl of 0.1 M HEPES buffer, pH 8.0. Ten μl of the solution was incubated in a total volume of 100 μl with 4 U of carboxylesterase (EC. 3.1.1.1, from rabbit liver) (Sigma E-2884). The samples were incubated at 37° C. for 1 hour. The presence of the expected reaction product, tri(ethylene glycol)monomethyl ether in the reaction mixture was determined by LC-MS using a C 18 column eluted with acetonitrile/water/0,1% formic acid and full scan detection in a narrow mass range.

### EXAMPLE 7

### 1,4,7-Tris(carboxymethyl)-10-(4-methyl)benzamide-3-phenoxyphosphate Ester)-1,4,7,10-tetraazacyclododecane

The title product is prepared by the following steps:

3-Hydroxbenzene-(4-(chloromethyl))benzamide

4-(Chloromethyl)benzoylchloride (1.89 g, 10 mmol) and triethylamine (2.02 g, 20 mmol) dissolved in DMF (40 ml) was added 3-hydroxyaniline (1.09 g, 10 mmol) over a period of 3 h at ambient temperature. The reaction mixture was stirred overnight and filtrated. The reaction mixture was evaporated in vacuo and submittet to flash chromatography (hexan/ethyl acetate, 1:1) giving the title compound. Yield: 1.51 g (61%). MS (EI):262 (9), [M+].

Diethyl 3-phenoxyphosphate-(4-(chloromethyl))benzamide

3-Hydroxbenzene-(4-(chloromethyl))benzamide (1.05 g,4 mmol) and triethylamine (1.21 g, 12 mmol) dissolved in THF (30 ml) was added diethyl chlorophosphate (2.07 g, 12 mmol) and stirred for 72 h. at ambient temperature. The reaction mixture was evaporated in vacuo and purified by flash chromatography (hexan/ethyl acetate, 1:1). Yield: 0.32 g (20%). MS (EI):399 (15), 397 (49) [M+].

1,4,7-Tris(tert-butylcarbonylmethyl)-10-((4-methyl)benzamide-3-phenoxyphosphate diethyl ester)-1,4,7,10-tetraazacyclododecane

Diethyl 3-phenoxyphosphate-(4-(chloromethyl))benzamide (1 eq.) is reacted with 1,4,7-Tris(tert-butylcarbonylmethyl)-1,4,7,10-tetraazacyclododecane (1 eq.) according to procedure (y) giving the title compound in good yield.

1,4,7-Tris(carboxymethyl)-10-((4-methyl)benzamide-3-phenoxyphosphate ester)-1,4,7,10-tetraazacyclododecane

1,4,7-Tris(tert-butylcarbonylmethyl)-10-((4-methyl)benzamide-3-phenoxyphosphate diethyl ester)-1,4,7,10-tetraazacyclododecane is added trifluoroacetic acid to remove the tert-butylgroup as earlier described before trimethylsilylbromide technology is used to selectively hydrolyse the ethylesters.

The Gd-chelate of the above product can easily be made by conventional methods. The product may be used in mapping of phosphatase activity in MRI.

### EXAMPLE 8

### Mapping Of Kinetics In Drug Metabolism Using Position Emission Tomography (PET).

Topotecan is an inhibitor of topoisomerase I which is a key enzyme for the integrity of DNA structure and thereby cell function. Topotecan is used to treat cancer; especially ovarian cancer and lung cancer.

¹¹C N-methyl radiolabelled topotecan can be prepared from N-desmethyl topotecan by methylation with ¹¹C-methyl iodide: N-desmethyl topotecan is prepared from topotecan using the same method as described by Rao, P. N. et al in Steroids, 64, 205-212 (1999). Radiolabelled topotecan was prepared by alkylation of N-desmethyl topotecan by ¹¹C-methyl iodide at 100° C. for about 4 minutes in dimethylformamide according to the method described in WO 01/21249 (Collins, J. M. et al).

Oxidative demethylation in vivo is mapped using PET to follow the rediction of the concentration of radiotracer in target tissue and/or redistribution/elimination of the radiotracer as formaldehyde, formic acid or derivatives thereof.

### EXAMPLE 9

### Mapping Of Kinetics In Drug Metabolism Using ¹³C-labelled Drugs And In Vivo ¹³C MR-spectroscopy And/or ¹³C MR Imaging.

Various drugs can be labelled with ¹³C in positions where the ¹³C-chemical shift in NMR is changed as a result of a metabolic transformation (¹³C close to position for metabolic transformation) or labelled with ¹³C so that the ¹³C-containing metabolite has different pharmacokinetic/pharmacodynamic properties than the parent drug.

The kinetics in metabolism can be followed using in vivo ¹³C MR-spectroscopy and/or ¹³C MR imaging.

Kinetics of metabolism can also be performed with fluorine-containing compounds and in vivo ¹⁹F MR spectroscopy and/or ¹⁹F-imaging.

The signal may also be enhanced by using hyperpolarizing techniques.

### EXAMPLE 10

### A Compound, Comprising A Chelator, That Is A Substrate For Transglutaminase.

The bis-anhydride of diethylene-triamine-pentaacetic acid (DTPA) is treated with an excess of the mono-benzyloxycarbonyl derivative of 1,6-diaminohexane in water at slightly alkaline pH. The excess of the latter compound is removed by adding an excess of benzyloxycarbonyl chloride, producing the water-insoluble bis-benzyloxycarbonyl derivative of 1,6-diamino-hexane. After the excess benzyloxycarbonyl chloride is decomposed, the reaction mixture is filtered. The benzyloxycarbonyl groups are removed by hydrogenation and the DTPA derivative (compound I) is further purified by ion exchange chromatography.

### EXAMPLE 11

### A Transglutaminase Substrate For Use In MRI

Gadolinium ions are complexed with the compound I above. The resulting chelate is injected into an experimental animal that has a condition that involves apoptosis. The animal may, for instance, be remodeling the mammary glands following cessation of lactation, or it may have a tumour. The transglutaminase in the apoptotic cells will covalently link the chelate to proteins by its aminohexane side chains, producing a large increase in relaxivity and also stopping the gadolinium complex from being washed out of the tissue. Apoptotic tissue in the animal is imaged by MRI.

### EXAMPLE 12

### A Transglutaminase Substrate For Use In Scintigraphy

^{99m}Tc ions are complexed with the compound I above. The resulting chelate is injected into an experimental animal that has a condition that involves apoptosis. The animal may, for instance, be remodeling the mammary glands following cessation of lactation, or it may have a tumour. The transglutaminase in the apoptotic cells will covalently link the chelate to proteins by its aminohexane side chains, stopping the technetium complex from being washed out of the tissue. Apoptotic tissue in the animal is imaged by scintigraphy.

### EXAMPLE 13

### A Compound, Comprising A Chelator, That Is A Substrate For Transglutaminase.

The bis-anhydride of diethylene-triamine-pentaacetic acid (DTPA) is treated with an excess of the peptide Gly-GlN-Gly at slightly alkaline pH. The DTPA derivative (compound II) is further purified by ion exchange chromatography.

### EXAMPLE 14

### A Transglutaminase Substrate For Use In MRI

Gadolinium ions are complexed with compound II above. The resulting chelate is injected into an experimental animal that has a condition that involves apoptosis (as above). The transglutaminase in the apoptotic cells will covalently link the chelate to proteins by its glutamine side chains, producing a large increase in relaxivity and also stopping the gadolinium complex from being washed out of the tissue. Apoptotic tissue in the animal is imaged by MRI.

### EXAMPLE 15

### A Transglutaminase Substrate For Use In Scintigraphy

^{99m}Tc ions are complexed with compound II above. The resulting chelate is injected into an experimental animal that has a condition that involves apoptosis (as above). The transglutaminase in the apoptotic cells will covalently link the chelate to proteins by its glutamine side chains, stopping the gadolinium complex from being washed out of the tissue. Apoptotic tissue in the animal is imaged by scintigraphy.

### EXAMPLE 16

### A Substrate For Caspase 3, Comprising A Chelator, That Alters Its Charge By The Action Of The Enzyme

The N-hydroxy-succinimide ester of the peptide Asp-Glu-Val-Asp is synthetized by conventional methods of peptide chemistry. It is added to compound I in 2.5-fold excess in aqueous solution at pH 8. The resulting bis-peptidyl derivative of compound I is purified by ion exchange chromatography (compound III).

### EXAMPLE 17

### A Substrate For Caspase 3 For Use In MRI

Gadolinium ions are complexed with compound III above. The resulting chelate is injected into an experimental animal that has a condition that involves apoptosis (as above). The caspase 3 in the apoptotic cells will cleave the acidic peptide from the aminohexyl side chains, changing the charge of the substrate from about −4 to about +2 in the product at approximately neutral pH. This promotes the association of the product with intracellular proteins, most of which are negatively charged. In turn, this produces an increase in relaxivity and also prevents the gadolinium complex from being washed out of the tissue. Apoptotic tissue in the animal is imaged by MRI.

### EXAMPLE 18

### A Caspase 3 Substrate For Use In Scintigraphy

^{99m}Tc ions are complexed with compound III above. The resulting chelate is injected into an experimental animal that has a condition that involves apoptosis (as above). The caspase 3 in the apoptotic cells will will cleave the acidic peptide from the aminohexyl side chains, changing the charge of the substrate from about −4 to about +2 in the product at neutral pH. This promotes the association of the product with intracellular proteins, most of which are negatively charged. In turn, the technetium complex is prevented from being washed out of the tissue. Apoptotic tissue in the animal is imaged by MRI.

### EXAMPLE 19

### A Transglutaminase Substrate For Use In MRI

The carboxylic acid groups of the compound 1,4,8,11-tetraazacyclotetradecane-1,4,8,11-tetraacetic acid are reacted with a five-fold molar excess of 1,6-diaminohexane in the presence of 1.5 molar equivalents of 1-(3-dimethylaminopropyl)-3-ethylcarbodiimide in water at pH 5. The excess of 1,6-diaminohexane,1-(3-dimethylaminopropyl)-3-ethylurea and remaining carbodiimide are removed by ion exchange chromatography (compound IV).

Gadolinium ions are complexed with compound IV above. The resulting chelate is injected into an experimental animal that has a condition that involves apoptosis (as above). The transglutaminase in the apoptotic cells will covalently link the chelate to proteins by its glutamine side chains, producing a large increase in relaxivity and also stopping the gadolinium complex from being washed out of the tissue. Apoptotic tissue in the animal is imaged by MRI.

### EXAMPLE 20

### A Transglutaminase Substrate For Use In MRI

The carboxylic acid groups of the compound 1,4,8,11-tetraazacyclotetradecane-1,4,8,11-tetraacetic acid are activated as N-hydroxysuccinimide esteres by reaction with N-hydroxysuccinimide and 1-(3-dimethylaminopropyl)-3-ethylcarbodiimide in water at pH 5 (compound V). The activated carboxyl groups are allowed to react with the peptide Gly-GlN-Gly (compound VI).

Gadolinium ions are complexed with compound VI above. The resulting chelate is injected into an experimental animal that has a condition that involves apoptosis (as above). The transglutaminase in the apoptotic cells will covalently link the chelate to proteins by its glutamine side chains, producing a large increase in relaxivity and also stopping the gadolinium complex from being washed out of the tissue. Apoptotic tissue in the animal is imaged by MRI.

### EXAMPLE 21

### A Caspase 3 Substrate For Use In MRI

The activated carboxyl groups of compound V are allowed to react with ethylene diamine. After removal of the excess ethylene diamine, the N-hydroxysuccinimide ester of the peptide Asp-Glu-Val-Asp is added to the compound. Finally, the protecting groups are removed by hydrogenation (compound VI).

Gadolinium ions are complexed with the compound above (compound VI). The resulting chelate is injected into an experimental animal that has a condition that involves apoptosis (as above). The caspase 3 in the apoptotic cells will cleave the acidic peptide from the amino-ethyl side chains, changing the charge from near neutral in the substrate to strongly positive in the product at approximately neutral pH. This promotes the association of the product with intracellular proteins, most of which are negatively charged. In turn, this produces an increase in relaxivity and also prevents the gadolinium complex from being washed out of the tissue. Apoptotic tissue in the animal is imaged by MRI.

### EXAMPLE 22

### A Microbubble Preparation That Is A Substrate For Matrix Metalloproteinase 7 (MMP-7)

Preparation of a undeca-lipid-derivatized peptide containing a MMP-7 cleavage site

The peptide BzlGlu-BzlGlu-BzlGlu-Ala-Pro-Leu-Gly-Leu-Leu-Ala-Arg (“BzlGlu” is glutamic acid esterified with benzyl alcohol at the 5-carboxyl group) is made by solid phase synthesis. The carboxyl terminus is activated as the N-hydroxysuccinimide ester by reaction with N-hydroxysuccinimide and dicyclohexylcarbodiimide. The activated peptide is reacted with distearoyl-phosphatidylethanolamine and the benzyl protecting groups are removed by hydrogenolysis. The reaction product is purified (compound A).

Preparation of microbubble dispersions by rotor stator mixing

500 mg of a mixture of Compound A and distearoylphosphatidylcholine in a mole ratio of 2:8 is added to 100 ml water containing 5.4% (w/w) of a mixture of propylene glycol and glycerol (3:10 w/w). The mixture is shaken and heated to 80° C. for five minutes, allowed to cool to room temperature, shaken again and left standing overnight prior to use. 50 ml of the resulting solution is transferred to a round-bottomed flask with a conical neck. The flask is fitted with a glass jacket having a temperature control inlet and outlet connected to a water bath maintained at 25° C. A rotor stator mixing shaft is introduced into the solution and to avoid gas leakage the space between the neck wall and the mixing shaft is sealed with a specially designed metal plug fitted with a gas inlet/ outlet connection for adjustment of gas content and pressure control. The gas outlet is connected to a vacuum pump and the solution is degassed for one minute. An atmosphere of perfluoro-n-butane gas is then applied through the gas inlet.

The solution is homogenised at 23000 rpm for 10 minutes, keeping the rotor stator mixing shaft such that the openings are slightly above the surface of the liquid. A white coloured creamy dispersion is obtained, which is transferred to a sealable container and flushed with perfluoro-n-butane. The dispersion is then transferred to a separating funnel and centrifuged at 12000 rpm for 30 minutes, yielding a creamy layer of bubbles at the top and a turbid infranatant. The infranatant is removed and replaced with water. The centrifugation is then repeated twice, but now at 12000 rpm for 15 minutes. After the last centrifugation, the supernatant is replaced by 10% (w/w) sucrose. Two ml portions of the resulting dispersion are divided between 10 ml flat-bottomed vials specially designed for lyophilisation, and the vials are cooled to −47° C. and lyophilised for approximately 48 hours, giving a white fluffy solid substance. The vials are now transferred to a vacuum chamber, and air is removed by a vacuum pump and replaced by perfluoro-n-butane gas. Prior to use, water is added and the vials are gently hand-shaken for several seconds, giving microbubble dispersions suitable as ultrasound contrast agents.

Characterisation

The size distribution and volume concentration of the microbubbles are measured using a Coulter Counter Mark II apparatus fitted with a 50 μm aperture with a measuring range of 1-30 μm. 20 μl samples are diluted in 200 ml saline saturated with air at room temperature, and allowed to equilibrate for 3 minutes prior to measurement.

Ultrasound characterisation is performed on a experimental setup slightly modified from de Jong, N. and Hoff, L. as described in “Ultrasound scattering properties of Albunex microspheres”, Ultrasonics 31(3), pp. 175-181 (1993). This instrumentation measures the ultrasound attenuation efficacy in the frequency range 2-8 MHz of a dilute suspension of contrast agent. During the attenuation measurement a pressure stability test is performed by exposing the sample to an overpressure of 120 mmHg for 90 seconds. Typically 2-3 μl of sample is diluted in 55 ml Isoton II and the diluted sample suspension is stirred for 3 minutes prior to analysis. As primary response parameter the attenuation at 3.5 MHz is used, together with the recovery attenuation value at 3.5 MHz after release of the overpressure.

Enzymatic generation of cationic charge

The microbubble suspension is incubated with 10 μg of recombinant MMP-7 in 50 mM tris-HCl buffer, pH 7.4, containing 10 mM CaCl₂ and 150 mM NaCl. The enzyme cleaves the peptide in the vicinity of the two neighbouring leucines, liberating a peptide bearing a net negative charge of 2 and leaving a net positive charge. The net positive charge allows the microbubbles to bind to cell surfaces or extracellular matrix close to the site of charge alteration by the enzyme.

The change in charge of the microbubbles in the dispersion is monitored using zeta-potential measurement in a Malvern Zetasizer 3000 HS equipped with an electrophoresis cell. The instrument is checked using the negatively charged latex standard DTSO050. For the measurements, 50 μl of microbubble suspension is diluted with 100 ml of 0.01% NaCl solution.

### EXAMPLE 23

### A Microbubble Preparation That Is A Substrate For Matrix Metalloproteinase 7 (MMP-7)

Preparation of a heptalipid-derivatized peptide containing a MMP-7 cleavage site

The peptide Pro-Leu-Gly-Leu-Leu-Ala-Arg is made by solid phase synthesis. The carboxyl terminus is activated as the N-hydroxysuccinimide ester by reaction with N-hydroxy-succinimide and dicyclohexylcarbodiimide. The activated peptide is reacted with distearoyl-phosphatidylethanolamine. Finally, the derivatized peptide is reacted with an excess of 1,2,4-benzenetricarboxylic anhydride. The reaction product is purified. The subsequent steps are the same as in the example above, one of the essential points being that cleavage of the peptide removes two negative charges and leaves the product with a net positive charge.

### EXAMPLE 24

### A Microbubble Preparation That Is A Substrate For Aminopeptidase A

Preparation of a lipid-derivatized substrate for aminopeptidase A

The peptide N-BzBzlGlu-Ala is made by chemical synthesis (“N-BzBzlGlu” is glutamic acid protected by the benzyloxycarbonyl group on the amino group and esterified with benzyl alcohol at the 5-carboxyl group). The carboxyl terminus is activated as the N-hydroxy-succinimide ester by reaction with N-hydroxysuccinimide and dicyclohexylcarbodiimide. The activated peptide is reacted with distearoyl-phosphatidylethanolamine and the protecting groups are removed by hydrogenolysis. The reaction product is purified (compound B).

Preparation of microbubble dispersions by rotor stator mixing

500 mg of a mixture of Compound B, distearoylphosphatidylglycerol and distearoyl-phosphatidylcholine in a mole ratio of 1.5:0.5:8 is added to 100 ml water containing 5.4% (w/w) of a mixture of propylene glycol and glycerol (3 :10 w/w). (The distearoylphosphatidylglycerol is added in order to prevent microubble aggregation by the presence of a small net charge.) The rest of the procedure for preparation of microbubble dispersions is as described above.

Enzymatic generation of cationic charge

One volume of the microbubble suspension is incubated with ten volumes of fresh serum. Aminopeptidase A removes the N-terminal glutamic acid, leaving a net positive charge of 1 (amino terminus). The net positive charge allows the microbubbles to bind to cell surfaces or extracellular matrix.

The change in charge of the microbubbles in the dispersion is monitored using Z-potential measurement in a Malvern Zetasizer 3000 HS equipped with an electrophoresis cell. The instrument is checked using the negatively charged latex standard DTSO050. For the measurements, aliquots are removed from the incubation with serum. In order to remove interfering protein, the microbubbles are flotated by brief centrifugation and re-suspended in the same volume of 0.01 % NaCl solution. 500 μl of microbubble suspension is diluted with 100 ml of 0.01% NaCl solution.

### EXAMPLE 25

### A Gelatinase-binding Peptide For Imaging Atherosclerotic Plaques. Contrast Agents For Imaging Atherosclerotic Plaques By MRI And Scintigraphy

Gelatinase is a metalloproteinase that is expressed in unstable atherosclerotic plaques. The cyclic peptide Cys-Thr-Thr-His-Trp-Gly-Phe-Thr-Leu-Cys was identified as a gelatinase inhibitor (Koivunen et al. (1999) Nature Biotechnol. 17, 768-74).

The peptide is synthetized by solid phase techniques and allowed to cyclise. About 2.5 molar equivalents of the peptide is reacted with 1 equivalent of the bis-anhydride of diethylene-triamine-pentaacetic acid (DTPA) in water at slightly alkaline pH (Compound C).

Gadolinium ions are complexed with the compound C. The resulting chelate is injected into an experimental animal that has experimental atherosclerosis, for instance, a cholesterol-fed rabbit or any of several strains of transgenic mice. The peptide-chelate will bind to gelatinase in the atherosclerotic plaques, which may be imaged by MRI.

^{99M}Tc ions are complexed with the compound C above. The resulting chelate is injected into an experimental animal that has experimental atherosclerosis, for instance, a cholesterol-fed rabbit or any of several strains of transgenic mice. The peptide-chelate will bind to gelatinase in the atherosclerotic plaques, which may be imaged by scintigraphy.

1. A contrast agent substrate susceptible of changing pharmacodynamic and/or pharmacokinetic properties upon the influence of enzymatic activity.

2. A contrast agent substrate of claim 1 wherein the change in properties involves a change from the contrast agent substrate to a contrast agent product.

3. A contrast agent substrate of claim 1 or 2 wherein the change from the contrast agent substrate to a contrast agent product involves a chemical modification.

4. A contrast agent substrate of claims 1-3 for detecting enzyme activity characterized in that the contrast agent substrate changes pharmacodynamic properties and/or pharmacokinetic properties upon a chemical modification of the contrast agent substrate to a contrast agent product upon a specific enzymatic transformation.

5. A contrast agent substrate of claims 1-4 for detection of an area of disease of abnormal enzymatic activity.

6. A contrast agent substrate of claims 1-5 for detection of tissue or cells with abnormal metabolic activity.

7. A contrast agent substrate of any of the claims 1-6 for identification and/or diagnosis of cancer, cardiovascular diseases, diseases on the central nervous system, inflammations or infections.

8. A contrast agent substrate of any of the claims 1-7 where the contrast agent substrate is an MRI contrast agent, a radiopharmaceutical contrast agent, an ultrasound contrast agent, an optical imaging contrast agent or an x-ray contrast agent.

9. A contrast agent substrate of any of the claims 1-7 where the contrast agent substrate is an MRI or radiopharmaceutical contrast agent.

10. A contrast agent substrate of any of the claims 1-7 where the contrast agent substrate is an MRI contrast agent.

11. A contrast agent substrate of any of the claims 1-10 characterized in that the contrast agent substrate comprises a contrast active element bound to an enzyme substrate, optionally the contrast active element and the substrate are linked by a spacer.

12. A contrast agent substrate of claim 11 wherein the contrast agent substrate further comprises a targeting vector.

13. A contrast agent substrate as claimed in claim 11 and 12 wherein the enzyme substrate:
a) is processed by the enzyme;
b) liberates the contrast active element attached to the targeting vector;
and wherein the targeting vector attached to the contrast active element is bound to a target/receptor in or around the diseased area and thus enhancing the binding of the contrast active element.

14. A contrast agent substrate of any of the claims 1-13 characterized in that the contrast agent substrate upon an enzymatic transformation changes binding properties to biological surfaces.

15. A contrast agent substrate of any of the claims 1-13 characterized in that the contrast agent substrate upon an enzymatic transformation results in a change in rate of penetration of biological membranes and/or in changes in membrane permeability and/or affinity for a transport protein.

16. A contrast agent substrate of any of the claims 1-15 characterised in that the enzymatic transformation modifying the contrast agent substrate to a contrast agent product involves one or more of the following enzymes; cyclooxygenase, farnesyltransferase, matrix metalloproteinases, topoismerase, telomerase, angiotensin, converting enzyme (ACE), Hydroxymethylglutaryl-CoA reductase, endothelial constitutive nitric oxide synthase, inducible nitric oxide synthase, nitric oxide synthase, endothelin converting enzyme, protein serine-threonine kinase, superoxide dismutase, thrombin, plasmin, plasminogen activator and lipoprotein lipase, protein kinases, monoamine oxydase, myelin basic protein kinase, glutamate translocase, tyrosine 3-monooxygenase, hydrolases, matrix protease and calpain, collagenases, RNA replicase, endopeptidase, DNA helicase, viral neuramidase, [HIV] reverse transcriptase, viral integrase and proteases, beta-lactamase, serine endopeptidase, muramidase, 1,3-beta-glucan synthase, calcineurin, chitin synthetase, glycylpeptide-N-myristoyl transferase, phosphatase, esterase, or glucosidase.

17. A contrast agent substrate of any of the claims 1-15 characterised in that the enzymatic transformation modifying the contrast agent substrate to a contrast agent product involves one or more of the following enzymes; cyclooxygenase, farnesyltransferase, matrix metalloproteinases, topoismerase, telomerase.

18. Use of a contrast agent substrate of any of the claims 1-16 for detecting an area of disease of abnormal enzymatic activity.

19. Use of a contrast agent substrate of any of the claims 1-17 for manufacturing of a medicament for detecting an area of disease of abnormal enzymatic activity.

20. A method for detection of abnormal enzymatic activity characterized in that a contrast agent substrate is administered to a human or animal body and a contrast agent signal is detected as a result of the contrast agent changing pharmacodynamic and/or pharmacokinetic properties upon the influence of enzymatic activity.
